(19) United States
(12) Patent Application Publication
(10) Pub. No.: US 2003/0082544 A1
(43) Pub. Date: May 1, 2003

(54) Methods and systems for validating detection assays, developing in-vitro diagnostic DNA or RNA analysis products, and increasing revenue and/or profit margins from in-vitro diagnostic DNA or RNA analysis assays

(75) Inventors: Lance Fors, Monrovia, CA (US); Rocky Ganske, Madison, WI (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 09/915,063

(22) Filed: Jul. 25, 2001

Related U.S. Application Data
(60) Provisional application No. 60/304,521, filed on Jul. 11, 2001.

(51) Int. Cl. G06F 17/60; C12Q 1/68; G01N 33/53; G06F 19/00; G01N 33/48; G01N 33/50

(52) U.S. Cl. 435/6; 435/7.1; 702/20; 705/2

(57) ABSTRACT

The present invention provides systems and methods for acquiring and analyzing biological information. For example, the present invention provides systems and methods for the use of genetic information in the generation of assays for detecting the genetic identity of samples, the production of assays, the use of assays for gathering genetic information of individuals and populations, and the storage, analysis, and use of the obtained information.

This application claims priority from U.S. Provisional Application filed Jul. 11, 2001 under Express Mail Label No. EL 790 816 448 US.

### FIELD OF THE INVENTION

The present invention provides systems and methods for acquiring and analyzing biological information. In particular, the present invention provides systems and methods for developing detection assays and for use of detection assays in basic research discovery to facilitate selection and development of clinical detection assays. As a detection assay is moved from research to clinical use, the cost to produce it does not increase significantly, while the revenue and profit margin it generates increase exponentially.

### BACKGROUND

As the Human Genome Project nears completion and the volume of genetic sequence information available increases, genomics research and subsequent drug design efforts increase as well. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations. However, despite the wealth of sequence information available, information on the frequency and clinical relevance of many polymorphisms and other variations has yet to be obtained and validated. For example, the human reference sequences used in current genome sequencing efforts do not represent an exact match for any one person's genome. In the Human Genome Project (HGP), researchers collected blood (female) or sperm (male) samples from a large number of donors. However, only a few samples were processed as DNA resources, and the source names are protected so neither donors nor scientists know whose DNA is being sequenced. The human genome sequence generated by the private genomics company Celera was based on DNA samples collected from five donors who identified themselves as Hispanic, Asian, Caucasian, or African-American. The small number of human samples used to generate the reference sequences does not reflect the genetic diversity among population groups and individuals. Attempts to analyze individuals based on the genome sequence information will often fail. For example, many genetic detection assays are based on the hybridization of probe oligonucleotides to a target region on genomic DNA or mRNA. Probes generated based on the reference sequences will often fail (e.g., fail to hybridize properly, fail to properly characterize the sequence at specific position of the target) because the target sequence for many individuals differs from the reference sequence. Differences may be on an individual-by-individual basis, but many follow regional population patterns (e.g., many correlate highly to race, ethnicity, geographic local, age, environmental exposure, etc.). With the limited utility of information currently available, the art is in need of systems and methods for acquiring, analyzing, storing, and applying large volumes of genetic information with the goal of providing an array of detection assay technologies for research and clinical analysis of biological samples.

### SUMMARY OF THE INVENTION

The present invention provides systems and methods for acquiring and analyzing biological information. In particular, the present invention provides systems and methods for developing detection assays and for use of detection assays in basic research discovery to facilitate selection and development of clinical detection assays.

In some embodiments, the present invention provides methods of validating a detection assay, comprising: a) collecting test result data from a plurality of users, wherein the test result data is generated with one or more detection panels, and wherein the detection panels comprise a plurality of candidate detection assays configured for target detection; and b) processing at least a portion of the test result data such that at least one valid detection assay is identified from the plurality of candidate detection assays. In other embodiments, the method further comprises step c) marketing said valid detection assay as an Analyte-Specific Reagent or an In-Vitro Diagnostic. In certain embodiments, said marketing comprises selling and/or advertising. In other embodiments, the present invention provides methods of validating a detection assay, comprising: a) distributing one or more detection panels to a plurality of users, wherein the detection panels comprise a plurality of candidate detection assays configured for target detection; b) collecting test result data from at least a portion of the plurality of users, wherein the test result data is generated with the detection panels; and c) processing at least a portion of the test result data such that at least one valid detection assay is identified from the plurality of candidate detection assays. In other embodiments, the method further comprises step d) marketing said valid detection assay as an Analyte-Specific Reagent or an In-Vitro Diagnostic. In certain embodiments, said marketing comprises selling and/or advertising.

In particular embodiments, the plurality of detection assays comprise two or more unique detection assays (e.g. 10, . . . 50, . . . 100, . . . 1000, or more unique detection assays). In some embodiments, the plurality of detection assays comprise two or more unique INVADER assays (e.g. 10, . . . 50, . . . 100, . . . 1000, or more unique INVADER assays).

In certain embodiments, the methods of the present invention further comprise a distribution system, wherein the distributing is accomplished with the distribution system. In some embodiments, the distributing one or more detection panels to the plurality of users is at a reduced cost. In other embodiments, the distributing one or more detection panels to the plurality of users is at a subsidized cost. In still other embodiments, the distributing one or more detection panels to the plurality of users is at no cost.

In certain embodiments, prior to step a), the method further comprises the step of employing one or more of the plurality of candidate detection assays to discover at least one single nucleotide polymorphism. In particular embodiments, the plurality of detection assays comprise INVADER assays. In other embodiments, prior to step a), the method further comprises the step of utilizing one or more of the plurality of candidate detection assays to associate a single nucleotide polymorphism with a medical condition. In certain embodiments, the plurality of detection assays comprise INVADER assay components. In some embodiments, prior to step a), the method further comprises the step of utilizing one or more of the plurality of candidate detection assays, and computer aided analysis, to associate a single nucleotide polymorphism with a medical condition. In certain embodiments, the plurality of detection assays comprise INVADER assay components. In other embodiments, the INVADER assay components comprise an INVADER oligonucleotide, a probe, and a control target sequence. In particular embodiments, the plurality of detection assays comprise TAQMAN assay components (e.g. a probe and control target sequence).

In some embodiments, the one or more detection panels are configured for detecting a marker associated with a disease category. In certain embodiments, the disease category is selected from cardiovascular disease, cancer, autoimmune disease, metabolic disorders, neurological disease, musculoskeletal disorders, and endocrine related diseases.

In certain embodiments of the methods of the present invention, the plurality of users comprise researchers. In other embodiments, the plurality of users comprises at least 10 individual users. In some embodiments, the plurality of users comprises at least 200 individual users. In particular embodiments, the plurality of users comprises at least 500 individual users. In still other embodiments, the plurality of users comprises at least 1000 individual users. In particular embodiments, the plurality of users comprises at least 10,000 individual users.

In some embodiments of the methods of the present invention, the plurality of detection assays comprises at least 10 unique detection assays. In other embodiments, the plurality of detection assays comprises at least 1000 unique detection assays. In particular embodiments, the plurality of detection assays comprises at least 10,000 unique detection assays. In certain embodiments, the plurality of detection assays comprises at least 50,000 unique detection assays.

In particular embodiments, the method further comprises a step, after the processing step, of selling the at least one valid detection assay as an Analyte Specific Reagent (ASR). In some embodiments the method further comprises a step, after the processing step, of selling the at least one valid detection assay as an Analyte Specific Reagent (ASR) to an In-Vitro Diagnostic Manufactuer or to a non-clinical laboratory. In additional embodiments, the method further comprises a step, after the processing step, of selling the at least one valid detection assay as an In-Vitro Diagnostic.

In some embodiments, the test result data comprises raw assay data. In other embodiments, test result data comprises analyzed assay data. In certain embodiments, the test result data comprises both raw assay data and analyzed assay data. In particular embodiments, the test result data comprises data resulting from testing of at least separate samples (e.g. at least 1000, at least 10,000, or at least 100,000 separate samples).

In certain embodiments, the collecting comprises receiving the test result data from at least a portion of the plurality of users over a communications network (e.g. Internet or World Wide Web). In some embodiments, the collecting further comprises storing the test result data in a database. In particular embodiments, the database is part of a computer system of a service provider. In certain embodiments, the collecting comprises receiving the test result data over the Internet. In some embodiments, the collecting comprises retrieving the test result data from a user's computer system over a communication network. In additional embodiments, the user's computer system comprises a software application configured to receive the test result data. In some embodiments, the software application is further configured to transmit the test result data automatically via a communications network.

In some embodiments, the processing comprises categorizing the test result data (e.g. arranging the data according to unique detection assay and/or type of medical condition associated with detection of a target). In other embodiments, the processing comprises in silico analysis. In certain embodiments, the processing comprises computer aided analysis of the test result data. In additional embodiments, the processing comprises mathematical manipulation of the test result data. In further embodiments, the processing comprises comparing the test result data to a substantially equivalent predicate assay. In particular embodiments, the processing comprises mathematical manipulation of the test result data, and comparing the test result data to a substantially equivalent predicate assay.

In certain embodiments, at least one valid detection assay is identified as a result of being substantially equivalent to a predicate assay. In some embodiments, processing at least a portion of the test result data generates assay validation information.

In some embodiments, the methods of the present invention further comprise step e) submitting the assay validation information to a government body charged with approving products for clinical use. In certain embodiments, the government body is the Food and Drug Administration. In particular embodiments, the assay validation information is part of a 510(k) application that is submitted to the Food and Drug Administration. In other embodiments, the methods of the present invention further comprise a step of receiving approval from the Food and Drug Administration to market the at least one valid detection assay as an FDA approved In-Vitro diagnostic assay. In additional embodiments, the FDA approved In-Vitro diagnostic assay is a predicate for determining substantially equivalency for other In-Vitro diagnostic assays.

In some embodiments, the target is a single nucleotide polymorphism (e.g. in a DNA or RNA molecule). In other embodiments, the target is RNA (e.g. such that RNA expression can be quantitated).

The present invention also provides a method of developing an in-vitro diagnostic DNA or RNA analysis product comprising, running an assay through a product development funnel, in which the assay that enters the product development funnel is substantially similar to the in-vitro diagnostic DNA or RNA analysis product. In some embodiments, the assay is an assay to detect a single nucleotide polymorphism. In some preferred embodiments, the product development funnel optionally comprises one or more of the following: a discovery portion, a medically associated portion, an analyte-specific reagent portion, and an in-vitro diagnostic portion. In some embodiments, the assay comprises a chromosome specific assay. In some embodiments, the method further comprises the step of using a panel, wherein the panel comprises the assay. In other embodiments, the panel comprises a whole genome panel.

In some embodiments, the medically associated portion of the funnel comprises a panel organized by disease. In some preferred embodiments, the panel organized by disease is selected from the group consisting of a cardiovascular disease panel, an oncology panel, an immunology panel, a metabolic disorders panel, a neurological disorders panel, a musculoskeletal disorders panel, an endocrinology panel, and a genetic disease panel.

In some embodiments, the method further comprises the step of using a panel, wherein the panel is a panel for a multiplicity of disease states and/or wherein the panel comprises a drug metabolizing enzyme panel.

The present invention further provides a method of increasing revenue and/or a profit margin from the development of an in-vitro diagnostic DNA or RNA analysis product comprising channeling an assay through a product development funnel, in which the assay is substantially similar to the in-vitro diagnostic DNA or RNA analysis product. In some embodiments, the in-vitro DNA or RNA analysis product comprises an FDA approved product. In some preferred embodiments, the product development funnel has an ingress and an egress, wherein the assay is one of at least several thousand assays which enter the ingress. In other embodiments, the assay is one of about several hundred assays which exit the egress as the in-vitro diagnostic DNA or RNA analysis product.

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows a schematic summary of the flow of detection assay development in the present invention from research products to clinical products.

FIG. 2 shows a schematic summary of the discovery phase of the diagram shown in FIG. 1.

FIG. 3 shows a schematic summary of the development of potential clinical markers phase of the diagram shown in FIG. 1.

FIG. 4 shows exemplary detection assay products from each phase of the diagram shown in FIG. 1.

FIG. 5 shows business revenue generation from products from each phase of the diagram shown in FIG. 1. The arrows showing revenue/margin per detection assay are not quantitative, but simply show a qualitative increase for each layer of the funnel.

FIG. 6 shows an overview of in silico analysis in some embodiments of the present invention.

FIG. 7 shows an overview of information flow for the design and production of detection assays in some embodiments of the present invention.

FIG. 8 shows a computer display of an INVADERCREATOR Order Entry screen.

FIG. 9 shows a computer display of an INVADERCREATOR Multiple SNP Design Selection screen.

FIG. 10 shows a computer display of an INVADERCREATOR Designer Worksheet screen.

FIG. 11 shows a computer display of an INVADERCREATOR Output Page screen.

FIG. 12 shows a computer display of an INVADERCREATOR Printer Ready Output screen.

FIG. 13 shows a computer display of an association database.

FIG. 14 shows a computer display of a Microsoft Excel worksheet having data received by export from an association database.

FIG. 15 shows a computer display of a plate viewer.

FIG. 16 shows a computer display of a window for providing terms for searching an association database.

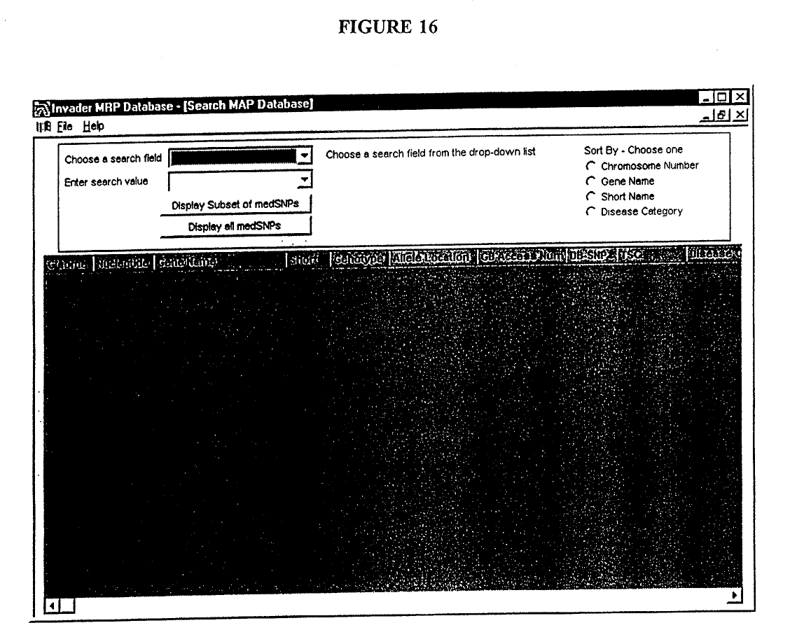

FIG. 17 shows a computer display of a data viewer.

FIG. 18 shows a computer display of allele caller results, having SNP results data displayed in the cells.

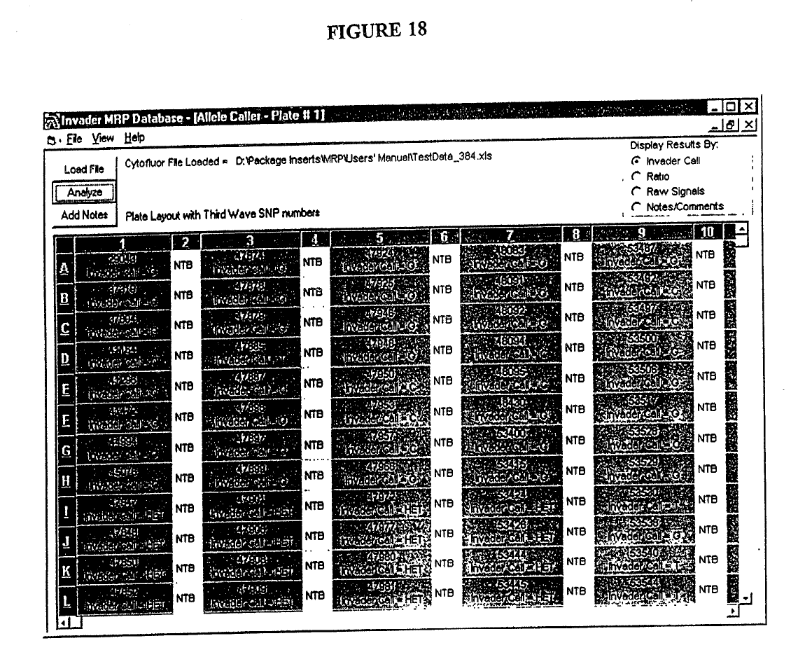

FIG. 19 shows a computer display of allele caller results, having analyzed input assay data (in this example, a calculated ratio) displayed in the cells.

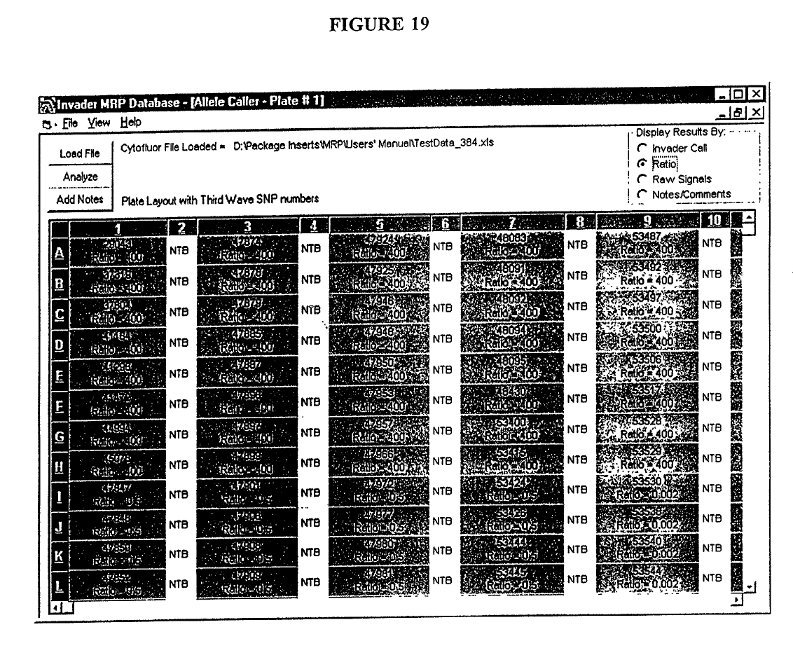

FIG. 20 shows a computer display of a Microsoft Excel worksheet having SNP results data received by export from an allele caller.

FIG. 21 shows an overview of the integration of components of the systems and methods of the present invention.

### GENERAL DESCRIPTION OF THE INVENTION

The present invention provides systems and methods for acquiring and analyzing biological information. In particular, the present invention provides systems and methods for developing detection assays and for use of detection assays in basic research discovery to facilitate selection and development of clinical detection assays.

The discovery of a new gene sequence suspected of correlating to a disease condition offers a starting point for understanding the correlation and hopefully, of leading to a treatment for the condition. However, extensive amounts of work need to be conducted before a useful and safe treatment can be obtained. The systems and methods of the present invention provide an efficient and thorough means to accelerate the time between initial discovery and useful treatment, and provide the tools for diagnosis and development of therapies. For example, where a mutation (e.g., a single nucleotide polymorphism; “SNP”) is suggested to correlate with a disease, the present invention provides systems for identifying an optimal target sequence from which an assay is developed to detect the presence of the mutation in a sample. The present invention also provides systems and methods for designing and producing a highly accurate detection assay directed to the optimized target sequence. The assay may then be used to detect the mutation in a large number of samples to determine the accuracy of the original proposed correlation and to determine additional information about the mutation (e.g., the allele frequency of the mutation in any desired population, data necessary for obtaining approval for clinical products from regulatory agencies, etc.). Data collected from these experiments is then analyzed and processed by systems and methods of the present invention to facilitate improved target selection, the identification of additional mutations, the identification of additional correlations, and the design of clinical assays for diagnosing the presence of the mutations in subjects (e.g., to identify subjects that are appropriate candidates for a particular type of therapy).

In some embodiments of the present invention, efficient, sensitive detection assays are provided. The assays are used by users (e.g. researchers) to collect test result data from a plurality of samples. Data obtained from the samples is used, among other purposes, to validate the detection assay. For example, collected test result data is used to provide evidence necessary to support approval (e.g., FDA approval) of clinical products corresponding to the detection assay. In some embodiments, a plurality of detection assays are combined into a panel and the panels are used to simultaneously collect data for multiple genetic markers. The collected data is used to provide evidence necessary to support approval of clinical products corresponding to one or more of the detection assays on the panel. In some embodiments, a party provides detection assays at a reduced cost, at a subsidized cost, or at no cost to users (e.g. researchers), and data collected by the users is used to support development and/or approval of clinical detection assay products by the providing party.

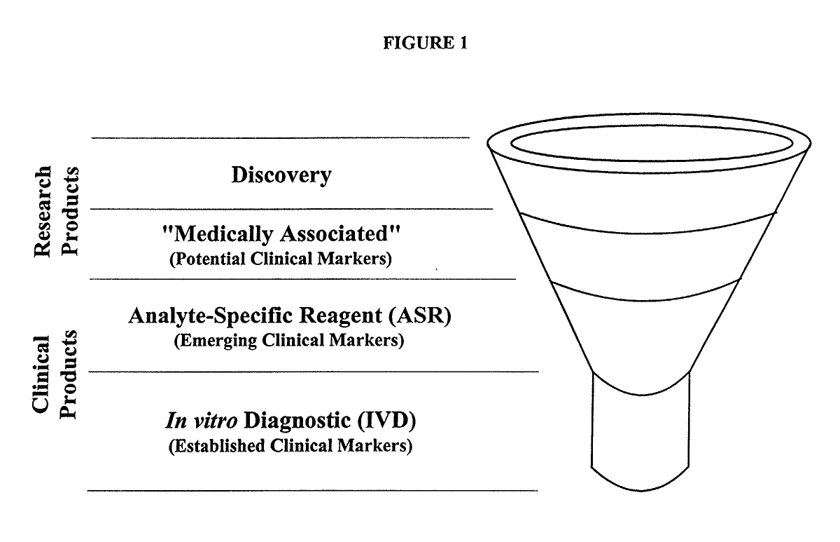

In some embodiments, the present invention provides systems and methods for the development of research and clinical diagnostic products using a multi-step process (i.e. product development funnel). A schematic summary of such a process is shown in FIG. 1. This figure shows four stages of detection assay development from discovery-based detection assays (e.g., identification and characterization of sequences and mutations), to medically associated marker detection assays (e.g., detection assays directed to markers associated directly or indirectly with one or more medically important conditions), to analyte-specific reagent assays, to clinical diagnostic detection assays (e.g., in vitro detection of established clinical markers). The funnel shown in FIG. 1 represents the fact that a large number of markers may be examined in the discovery phase, leading to a sub-set that are appropriate for each of the subsequent phases.

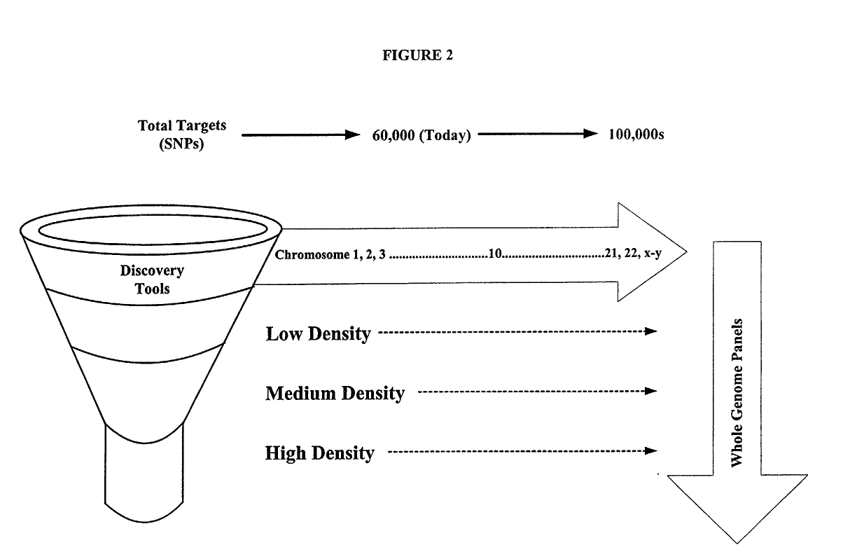

The following section describes how DNA analysis products directed to SNP detection are moved through the funnel. The focus on DNA products and SNP detection is for clarity only. RNA analysis products and other analysis products also find use in the present invention (e.g., for detecting and quantitating gene expression and other RNA levels using the same product strategy, including detection of splice variants and polymorphism variants). FIG. 2 shows a schematic summary of the discovery phase. In this phase detection assays directed to the thousands to hundreds of thousands of markers are generated. While the association of certain SNPs to particular medical conditions has been determined, association has not been established for the majority of SNPs. The present invention provides a broad menu of assays. For example, more than 50,000 unique assays applying the INVADER assay technology (Third Wave Technologies, Madison, Wis.) have been developed, manufactured and shipped for genotyping research to associate specific SNPs with predisposition to disease. Many of the assays have been sent to collaborative customers at low cost in exchange for access to collected data and rights to commercialize discoveries made with these collaborators.

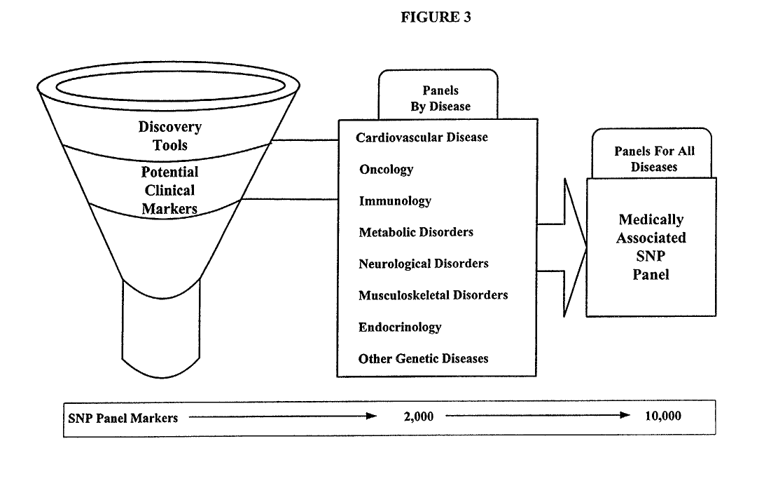

FIG. 3 shows a schematic summary of the “Medically Associated” phase. As use of detection assays reveals the potential association of a SNP with a medical condition, it is designated a potential clinical marker and earmarked for inclusion on one or more Medically Associated Panels (e.g., panels comprising a plurality of detection assays directed at two or more distinct markers). Using this approach, the association of certain SNPs has been established and panels have been prepared. New makers are added to panels as those markers are associated and moved down the funnel. FIG. 3 shows two types of panels, those containing markers specific to certain disease types or fields (e.g., cardiovascular disease, oncology, immunology, metabolic disorders, neurological disorders, musculoskeletal disorders, endocrinology, and other genetic diseases) and large panels (e.g., containing 10 thousand or more markers) directed to all known medically relevant diseases.

In one variant, researchers using the panels validate the associations of particular genetic markers to specific medical conditions Analyte-Specific Reagents (ASRs) phase). Once an association is valid, the assay is moved one step further down the funnel and, more importantly, into the clinical market. The ASR format permits the use of the assay in clinical settings without full FDA approval as the user, a certified clinical laboratory, validates the assay for the particular use. The format also allows for the generation of demand for a clinical marker or set of markers prior to deciding to seek FDA approval to market it as a in-vitro diagnostic tool.

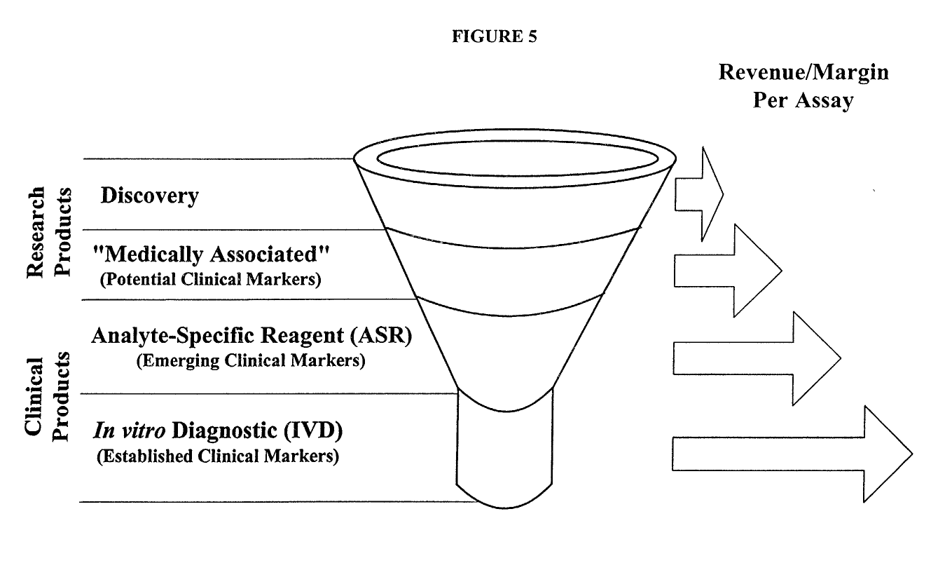

In yet another variant, which may include a Diagnostics phase, once sufficient market demand exists for a particular assay, full regulatory approval is sought to market the assay as an in vitro diagnostic (IVD). While IVD products are represented as occupying the smallest part of the funnel, they are the largest potential revenue source, as shown schematically in FIG. 5. As a detection assay is moved from research to clinical use, the cost to produce it does not increase significantly, while the revenue and profit margin it generates increase exponentially. The assay manufactured and shipped as an IVD is fundamentally the same assay that entered the top of the funnel as a discovery tool (although improvements or changes may be made during the process, as described below).

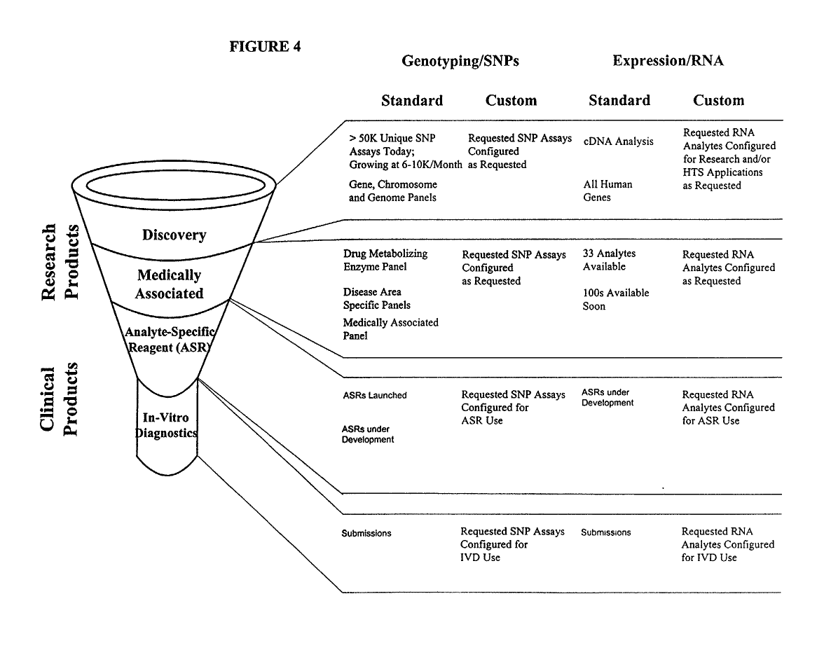

Examples of products for each of the funnel phases is shown in FIG. 4 for both genotyping and SNP detection of DNA samples (e.g., samples containing genomic DNA) and expression analysis. For the discovery phase, the systems and methods of the present invention have been applied to generate over 50 thousand unique SNP detection assays with the ability to add six to ten thousand additional unique SNP detection assays per month. In some embodiments, discovery panels are provided that are directed to SNP analysis of entire genes or chromosomes. The present invention also provides systems and methods for custom design of detection assays at any phase of the funnel (i.e., custom design of research and clinical detection assays). For the medically associated phase, specific panels have been developed for DNA analysis and a large number of expression analysis detection assays have been developed or are in development. For custom panel, customers may elect one or more markers of their choosing for use on the panel. In some embodiments, customers enter their desired panel components into a user interface of a software program and the received data is sent for analysis and production as described below.

In some embodiments, the funnel process is facilitated by a low cost, easy-to-use assay (e.g., the INVADER assay) and a production process that allows substantial numbers of detections assays to be generated. Such assays provide the necessary features (e.g., accuracy, sensitivity, ease-of-use, amenability to high throughput automated analysis, etc.) to allow wide-spread use by researchers, such that sufficient data is collected to process large numbers of detections assays through the funnel process. Widespread data collection results in the assay becoming a standard for use in discovery of the genetic basis of disease and management of personalized medicine strategies. For example, the present invention provides systems and methods to allow regulatory approval of clinical diagnostic products of every suitable marker.

Definitions

To facilitate an understanding of the present invention, a number of terms and phrases are defined below:

As used herein, the terms “SNP,” “SNPs” or “single nucleotide polymorphisms” refer to single base changes at a specific location in an organism's (e.g., a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCA1 gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “insilico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., diarrhea, nausea, fever, pain, and inflammation etc).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or DATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al, Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

As used herein, the term “distribution system” refers to systems capable of transferring and/or delivering materials from one entity to another or one location to another. For example, a distribution system for transferring detection panels from a manufacturer or distributor to a user may comprise, but is not limited to, a packaging department, a mail room, and a mail delivery system. Alternately, the distribution system may comprise, but is not limited to, one or more delivery vehicles and associated delivery personnel, a display stand, and a distribution center. In some embodiments of the present invention interested parties (e.g., detection panel manufactures) utilize a distribution system to transfer detection panels to users at no cost, at a subsidized cost, or at a reduced cost.

As used herein, the term “at a reduced cost” refers to the transfer of goods or services at a reduced direct cost to the recipient (e.g. user). In some embodiments, “at a reduced cost” refers to transfer of goods or services at no cost to the recipient.

As used herein, the term “at a subsidized cost” refers to the transfer of goods or services, wherein at least a portion of the recipient's cost is deferred or paid by another party. In some embodiments, “at a subsidized cost” refers to transfer of goods or services at no cost to the recipient.

As used herein, the term “at no cost” refers to the transfer of goods or services with no direct financial expense to the recipient. For example, when detection panels are provided by a manufacturer or distributor to a user (e.g. research scientist) at no cost, the user does not directly pay for the tests.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “derived from a different subject,” such as samples or nucleic acids derived from a different subjects refers to a samples derived from multiple different individuals. For example, a blood sample comprising genomic DNA from a first person and a blood sample comprising genomic DNA from a second person are considered blood samples and genomic DNA samples that are derived from different subjects. A sample comprising five target nucleic acids derived from different subjects is a sample that includes at least five samples from five different individuals. However, the sample may further contain multiple samples from a given individual.

As used herein, the term “treating together”, when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to a databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “PUSH technology” refers to an information dissemination technology used to send data to users over a network. In contrast to the World Wide Web (a “pull” technology), in which the client browser must request a Web page before it is sent, PUSH protocols send the informational content to the user computer automatically, typically based on information pre-specified by the user.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assays configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. medical condition). Examples of valid detection assays include, but are not limited to, detection assays that, when a target is detected, accurately predict the phenotype medical 95%, 96%, 97%, 98%, 99%, 99.5%, 99.8%, or 99.9% of the time. Other examples of valid detection assays include, but are not limited to, detection assays that quality as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

### DETAILED DESCRIPTION OF THE INVENTION

The following discussion provides a description of certain preferred illustrative embodiments of the present invention and is not intended to limit the scope of the present invention. For convenience, the discussion focuses on the application of the present invention to the detection of DNA targets, but it should be understood that the methods and systems are intended for use in the development of tools for the analysis of any nucleic acid analyte, e.g., DNA or RNA. Also, for the sake of illustration, the discussion often focuses on the characterization of SNPs using INVADER assay technology. It should be understood that the methods and systems of the present invention are intended for use in detecting other biologically relevant factors using a wide variety of detection assay technologies.

As discussed above, the present invention provides systems and methods for developing detection assays for research and clinical use. The following sections describe the high through-put design, optimization, and production of detection assays in a manner that allows assays to pass from a discovery phase to use as clinical diagnostic assays. The description is provided in the following sections: A) Detection Assay Development, Production, and Optimization; B) Development of Clinical Detection Assays; and C) Distribution and Use of Detection Assays.

A. Detection Assay Development, Production, and Optimization

The ability to detect the presence or absence of specific target sequences in a sample underlies much of the fields of molecular diagnostics and molecular medicine. For example, tremendous effort has been expended in the development of detection assays for nucleic acid sequence mutations that correlate to phenotypes of interest (e.g., inherited diseases).

The present invention provides systems and methods for acquiring and analyzing large amounts of biological information. For example, the present invention provides systems and methods for the use of genetic information in the generation of assays for detecting the genetic identity of samples, the production of assays, the use of assays for gathering genetic information of individuals and populations, and the storage, analysis, and use of the obtained information, including the use of information in selecting detection assays for research use, use in panels, use as ASRs, and use in clinical diagnostics (e.g., in-vitro diagnostics).

In some preferred embodiments, the present invention provides systems and methods for analyzing available sequence information (e.g., publicly available sequence information and information obtained by the methods described herein) in the selection of informative DNA and RNA target sequences for detections and analysis of individuals and populations. The present invention also provides systems and methods for the design and production of detection assays directed to such target sequences. The present invention further provides systems and methods for the collection, storage and analysis of data derived from detection assays. Importantly, the present invention provides integrated systems and methods that exploit the synergies of the above systems and methods to provide comprehensive solutions, allowing for large scale and informative analysis of sequences for identifying genotype/phenotype correlations, measuring differences in gene expression, identifying allele frequencies in populations, and typing individuals and populations for important (e.g., medically relevant) sequences. For example, in some embodiments, the present invention applies data obtained from detection assays to improve the selection of target sequences, design of improved assays, and selection of assays that are suitable for use on multi-analyte panels, as ASRs, and for clinical diagnostics.

The detection assay development, production, and optimization is illustrated below for hybridization-bases assays. One skilled in the art will appreciate the general applicability of various aspects of this description to other types of detection assays. The discussion of detection assay development, production, and optimization is provided in the following sections: I) Target Sequence Selection; II) Detection Assay Design; III) Detection Assay Production; IV) Detection Assay Use and Data Generation and Collection; and V) Integrated Information, Design, and Production (Optimization). It will be appreciated that every step may not be required for each detection assay. For example, where a valid target sequence and assay design are already known, production and testing may be started directly. The steps may be used for original assay development and/or may be used to re-evaluate a pre-existing detection assay, whether is be for a research or a clinical detection assay.

I. Target Sequence Selection

The ability to detect the presence or absence of specific target sequences in a sample underlies much of the fields of molecular diagnostics and molecular medicine. For example, tremendous effort has been expended in the development of detection assays for nucleic acid sequence mutations that correlate to phenotypes of interest (e.g., inherited diseases). During the development of the present invention, it was found that the design of a detection assay based on a published target sequence was often not sufficient to produce viable assays. In some circumstances assays will not work at all. In others, they may work for particular individuals or populations, but fail with other individuals or populations. The present invention provides systems and methods for selecting appropriate target sequences that can be successfully targeted by detection assays.

The problem with existing methods and the solutions provided by the present invention can be illustrated by example. Many detection assays are based on the principle of nucleic acid hybridization. An oligonucleotide is designed to hybridize to a portion of the target sequence; the presence of the hybrid, or the cleavage, elongation, ligation, disassociation, or other alterations of the oligonucleotide are detected as a means for characterizing the presence or absence of the sequence of interest (e.g., a SNP). Because there is sequence heterogeneity in the population, an oligonucleotide designed to hybridize to a target sequence of one individual may not hybridize to the corresponding sequence from another individual. For example, a first individual may have a gene sequence containing a SNP that is to be detected. A second individual may have the SNP, but also may have additional sequence differences in the vicinity of the SNP that prevent the hybridization of an oligonucleotide that was designed based on the sequence of the first individual. Additionally, target sequence information obtained from a public source may contain errors (e.g., may provide the wrong sequence) or may comprise incomplete, but essential, information. For example, a given target sequence may be found in multiple locations in the genome—the intended region that the assay is designed to detect, and unintended regions that would result in false positive or otherwise misleading assay results.

The systems and methods of the present invention provide an analysis of candidate target sequences to determine if they are suitable for use in detection assays. The systems and methods of the present invention also select appropriate sequences that are likely to function in the intended detection assay. This aspect of the present invention is referred to herein as “in silico analysis,” as computer analysis is conducted to analyze candidate target sequences against sequence and sequence-related information databases. In silico analysis may be performed prior to, or in conjunction with other processes of the present invention (e.g., detection assay design and production, selection of materials for panels, ASRs, and clinical tests, etc.).

In silico analysis methods of the present invention include one or more of the following sequence analysis and processing steps: input of a candidate sequence; editing of the candidate sequence, where necessary; screening of the candidate sequence for repeat sequences; screening of the candidate sequence for research artifact sequences; identification of the candidate sequence in a sequence database; conformation of the candidate sequence in a second (or additional) sequence database; information gathering using one or more sequence information databases; problem reporting; and/or transmission of an approved target sequence for production (e.g., automated production).

A. Sequence Input

Sequences may be input for in silico analysis from any number of sources. In many embodiments, sequence information is entered into a computer. The computer need not be the same computer system that carries out in silico analysis. In some preferred embodiments, candidate target sequences may be entered into a computer linked to a communication network (e.g., a local area network, Internet or Intranet). In such embodiments, users anywhere in the world with access to a communication network may enter candidate sequences at their own locale. In some embodiments, a user interface is provided to the user over a communication network (e.g., a World Wide Web-based user interface), containing entry fields for the information required by the in silico analysis (e.g., the sequence of the candidate target sequence). The use of a Web based user interface has several advantages. For example, by providing an entry wizard, the user interface can ensure that the user inputs the requisite amount of information in the correct format. In some embodiments, the user interface requires that the sequence information for a target sequence be of a minimum length (e.g., 20 or more, 50 or more, 100 or more nucleotides) and be in a single format (e.g., FASTA). In other embodiments, the information can be input in any format and the systems and methods of the present invention edit or alter the input information into a suitable form for in silico analysis. For example, if an input target sequence is too short, the systems and methods of the present invention search public databases for the short sequence, and if a unique sequence is identified, convert the short sequence into a suitably long sequence by adding nucleotides on one or both of the ends of the input target sequence. Likewise, if sequence information is entered in an undesirable format or contains extraneous, non-sequence characters, the sequence can be modified to a standard format (e.g., FASTA) prior to further in silico analysis. The user interface may also collect information about the user, including, but not limited to, the name and address of the user. In some embodiments, target sequence entries are associated with a user identification code.

In some embodiments, sequences are input directly from assay design software (e.g., the INVADERCREATOR software described below).

In preferred embodiments, each sequence is given an ID number. The ID number is linked to the target sequence being analyzed to avoid duplicate analyses. For example, if the in silico analysis determines that a target sequence corresponding to the input sequence has already been analyzed, the user is informed and given the option of by-passing in silico analysis and simply receiving previously obtained results.

B. In silico Processing Systems

In silico analysis utilizes one or more sequence and information databases (e.g., public or private sequence databases) and software applications for processing sequence and database information. In some preferred embodiments, databases and software for in silico analysis are housed in a single location on one or more computers. Housing the databases and processing software locally provides increased and consistent speed and access to information. In other embodiments, one or more databases and software components located on external computers are accessed over a communication network (e.g., accessed over the World Wide Web).

In preferred embodiments, databases that are maintained locally are updated regularly (e.g., following each update of the web-based server, a new version is downloaded to local servers). In some preferred embodiments, databases are surveyed periodically to determine if a new version is available and, if so, one is downloaded. In some preferred embodiments, more than one copy of each database is available locally. In particularly preferred embodiments, downloaded data is parsed to extract the data, and the parsed data is configured to automatically populate the fields of one or more receiving databases (e.g., an association database, a SNP database). In some embodiments, Perl scripts are used to sort data, e.g., line-by-line, and to create new text files (e.g., having data tagged according to the receiving field in the receiving database) for importation into the fields of a receiving database.

In some embodiments, the database analysis system comprises one or more central nodes (e.g., a computer containing a processor and computer memory) and a plurality of sub-nodes. In some embodiments, the sub-nodes house individual databases (or portions thereof) or software programs. In preferred embodiments, the central node controls the flow of information between sub-nodes, sending search requests to the sub-nodes and receiving search results from the sub-nodes. For example, in some embodiments, the central node directs data (e.g., candidate target sequence) to a sub node for a database search, receives the results, and directs the information to another sub-node for additional database searching. In some preferred embodiments, the central node directs information to multiple sub nodes simultaneously (e.g., for multiple concurrent database searches).

In some embodiments, in order to increase database access speed, individual databases are split among multiple (e.g., two) sub-nodes. In other embodiments, databases are housed on a single node. In preferred embodiments, databases are present in multiple copies on multiple sub-nodes. In some preferred embodiments, the central node monitors database load and status on each sub-node and directs searches to the node with the greatest available capacity.

In some preferred embodiments, the central node further directs resource management software. For example, individual nodes are sent test sequences on a regular basis to ensure that they are receiving information and processing information on a desired time scale. If a sub node is found to not be functioning properly, the central node directs information to a secondary sub node containing a copy of the database. In other embodiments, sub-nodes conduct self-monitoring routines and send status reports back to the central node. For example, in some embodiments, if a search on a sub-node fails or times out, the sub-node reports this information back to the central node so that appropriate action can be taken (e.g., send the search to another node and/or flag a particular sub-node for intervention). In some preferred embodiments, the central node maintains a queue of jobs submitted to each sub-node and warns human supervisors if a job fails to be completed.

In some embodiments, the central node comprises one or more workstations. In some embodiments, the sub nodes comprise two or more workstations. In other embodiments, the sub nodes comprise 5 or more workstations. In yet other embodiments, the sub nodes comprise 10 or more workstations. The present invention is not limited to a particular model or type of workstation. One skilled in the art understands that a variety of new processors of increasing speeds are regularly introduced into the market and that any suitable work station may be substituted for those described herein.

In some embodiments, in silico analysis of a candidate target sequence is completed in less than 10 seconds. In some preferred embodiments, in silico analysis of a candidate target sequence is completed in less than 2 seconds. In still more preferred embodiments, in silico analysis is completed in less than one second. In some embodiments, more than one (e.g., at least 5, preferably at least 20, and even more preferably, at least 100) sequences are analyzed simultaneously using the in silico analysis system of the present invention.

C. Preliminary Sequence Screening

In some embodiments of the present invention, the first step of in silico analysis of candidate target sequences is prescreening the candidate target sequences to maximize sequence database search efficiency.

In some embodiments, candidate target sequences are searched for repeat sequences. “Repeat sequences” refers to sequences that are known to repeat multiple times in a sample (e.g., in an organism's genome). Many genomes contain large regions of repeated sequences. The presence of repeated sequences in detection assay hybridization oligonucleotides can cause the oligonucleotide to hybridize to sequences other than, and/or in addition to, the intended target. Additionally, because repeat sequences are found in multiple copies in the genome, databases searches may operate very slowly or may not proceed. In some embodiments, RepeatMasker (available from at the public ftp site provided by Washington University, St. Louis, Mo.) is used to screen for repeat sequences. Repeat Masker screens DNA sequences for interspersed repeats and low complexity DNA sequences. Sequence information in FASTA format is input through a web-browser interface or by uploading a file. Multiple sequences may be input at once or may be contained within a file. There is no limit to the length of the query sequence or size of the batch file. Sequence comparisons in RepeatMasker are performed by the program Cross-match, an implementation of the Smith-Waterman-Gotoh algorithm developed by Phil Green. In some embodiments, RepeatMasker is run using MaskerAid (Bioinformatics 16:1040-1 [2000], available through licensing from Washington University in Saint Louis, Mo.), a performance enhancer for RepeatMasker. Execution profiling of native RepeatMasker showed that the vast majority of its time was spent running Cross-Match. MaskerAid allows the faster WU-BLAST search engine to substitute transparently for CrossMatch, yielding speed improvement while effectively maintaining sensitivity. MaskerAid is fundamentally a software “wrapper” around WU-BLAST that makes it appear and function very much like CrossMatch.

The output of the program is an annotation of the repeats that are present in the sequence of interest as well as a modified version of the sequence in which all the annotated repeats have been masked. The program returns three or four output files for each query. One contains the submitted sequence(s) in which all recognized interspersed or simple repeats have been masked. In the masked areas, each base is replaced with an N, so that the returned sequence is of the same length as the original. A table annotating the masked sequences as well as a table summarizing the repeat content of the query sequence is returned. Optionally, a file with alignments of the query with the matching repeats is returned as well.

Regions of low complexity, like simple tandem repeats, polypurine and AT-rich regions can lead to spurious matches in database searches. By default they are masked along with the interspersed repeats. With the option “Do not mask simple . . . ” only interspersed repeats are masked. This may, for example, be preferred in some embodiments where the masked sequence will be analyzed by a gene prediction program. Alternatively, with the option “Only mask simple . . . ”, one can mask only the low complexity regions (e.g., in some embodiments in which it is desirable to quickly locate polymorphic simple repeats in a sequence).

When checked, the repeat sequences are replaced by Xs instead of Ns. This allows one to distinguish the masked areas from possibly existing ambiguous sequences or other stretches of Ns in the original sequence. In some embodiments the use of X, N, or both may be desired for compatibility with database search engines used in the subsequent steps of the in silico analysis. In some embodiments, only the masked candidate target sequence is used in further in silico analysis. In other embodiments, both the masked and unmasked sequences are used in subsequent searches.

In certain cases, a majority or the entirety of the candidate target sequence may be masked by RepeatMasker. When this occurs, in some embodiments, a warning is sent to the user indicating that a potentially undesirable amount of the target sequence comprises repeat sequence. The user is then give the option of selecting a different target sequence or proceeding with the original sequence (or electing both options). When a decision to proceed with the sequence is selected, an unmasked version of the sequence is processed through the remaining in silico analysis steps. Where there is a portion of the original candidate target sequence that is not masked, both unmasked and masked sequences may be processed through the remaining in silico analysis steps. In some embodiments, in silico analysis is discontinued and the candidate target sequence is sent to production (Section III, below).

In some embodiments, prior to screening for repeat sequences, an analysis is performed to determine if the candidate target sequence contains undesired artifact sequences. For example, a number of sequences deposited in public databases contain vector sequence or other sequence artifacts as a result of molecular biology handling during their initial isolation and characterization. These artifact sequences often represent synthetic sequences not corresponding to a genome sequence, or inappropriately corresponding to a genome sequence other than the intended target. Where candidate target sequences are selected that contain artifact sequences, they are more likely to fail in detection assays and are more likely to result in undesirably long search times during the remaining in silico analysis steps. For example, rather than representing a sequence that appears once in a human genome, artifact sequence may correspond to thousands of deposited database sequence that each mistakenly contain a common vector sequence.

To correct for artifact sequence, in some embodiments, the present invention employs VecScreen (available at the National Center for Biotechnology Information, National Library of Medicine, National Institutes of Health public web site). VecScreen provides a system for identifying segments of a nucleic acid sequence that may be of vector origin. VecScreen searches a query for segments that match any sequence in a specialized non-redundant vector database (UniVec). The search uses a BLAST search routine with parameters preset for optimal detection of vector contamination. Those segments of the query that match vector sequences are categorized according to the strength of the match, and their locations are displayed.

The sequence of any vector contamination should theoretically be identical to the known sequence of the vector. In practice, occasional differences are expected to arise from sequencing errors, and less frequently, from engineered variants or spontaneous mutations. The search parameters used for VecScreen are chosen to find sequence segments that are identical to known vector sequences or which deviate only slightly from the known sequence. Vector containing sequences identified are then masked.

In some embodiments, the Repeat Masker and VecScreen screening are combined into a single search. In preferred embodiments, the candidate target sequence is first screened by VecScreen, with the results then passed through Repeat Masker. Once the screening is complete, masked sequences and/or unmasked sequences are ready for database searching as described below.

D. Database Searches

In some embodiments, database searches are performed on the candidate target sequences. Databases searches are used, among other purposes, to confirm that 1) the candidate target sequence is a sequence corresponding to a known sequence, 2) the candidate target sequence corresponds to a unique sequence in the sample to be tested, and 3) the candidate target sequence corresponds to a reliable (e.g., confirmed) sequence. The database searches are also used to gather information (allele frequencies, disease associations, variants, location in a genome, associated patents and patent applications, etc.) about the candidate target sequence. In some embodiments, the output information from the database searches is stored in a file associated with the candidate target sequence.

The present invention is not limited to the databases disclosed herein. Any database that provides relevant information may find use in the searches of the present invention. In some embodiments, searches are performed consecutively. In other embodiments, searches are performed concurrently. In preferred embodiments, some searches are performed consecutively and others are performed concurrently. In some embodiments, searches are performed using BLAST (Basic Local Alignment Search Tool) search mode using FASTA formatted sequences. In preferred embodiments, results from database searches are output as text files. Results are then converted to a format that is suitable for import into an Oracle database. In some embodiments, the BioJava Project is used to convert text output into an XML-like stream that is then incorporated into an Oracle database.

Descriptions of several databases that are searched in preferred embodiments of the present invention are described below.

1. SNP Databases

In preferred embodiments, candidate target sequences are first used to search a SNP database (e.g., including but not limited to dbSNP (National Human Genome Research Institute and NCBI)). The dbSNP database serves as a central repository for both single base nucleotide substitutions and short deletion and insertion polymorphisms. The data in dbSNP is integrated with other NCBI genomic data. If a match is found in the dbSNP, the output from the search is a dbSNP accession number. In some embodiments, the dbSNP search returns an accession # with an RS designation. This designation indicates that the SNP is a unique SNP identified as common between multiple studies.

2. Gene Loci Analysis

In some embodiments, following dbSNP searches, gene loci databases (e.g., Locus Link) are searched. LocusLink provides a single query interface to curated sequence and descriptive information about genetic loci. It presents information on official nomenclature, aliases, sequence accessions, phenotypes, EC numbers, MIM numbers, UniGene clusters, homology, map locations, and related web sites. The information output from LocusLink includes a LocusLink accession number (LocusID), an NCBI genomic contig number (NT#), an OMIM accession number, and a Unigene accession number (HS#).

3. Disease Association Databases

Following the LocusLink search, the information returned is used to search disease association databases. In some embodiments, the HUGO Mutation Database Initiative, which contains a collection of links to SNP/mutation databases for specific diseases or genes, is searched.

In some embodiments, the OMIM database is searched. OMIM (Online Mendelian Inheritance in Man) is a catalog of human genes and genetic disorders developed for the World Wide Web by NCBI, the National Center for Biotechnology Information. The database contains textual information and references. Output from OMIM includes a modified accession number where multiple SNPs are associated with a genetic disorder. The number is annotated to designate the presence of multiple SNPs associated with the genetic disorder.

4. Gene Oriented Cluster Analysis

In some embodiments, following dbSNP searches, software (e.g., including but not limited to, UniGene) is used to partition search results into gene-oriented clusters. UniGene is a system for automatically partitioning GenBank sequences into a non-redundant set of gene-oriented clusters. Each UniGene cluster contains sequences that represent a unique gene, as well as related information such as the tissue types in which the gene has been expressed and map location. In addition to sequences of well-characterized genes, hundreds of thousands novel expressed sequence tag (EST) sequences are included in UniGene. Currently, sequences from human, rat, mouse, zebrafish and cow have been processed.

Unigene can be searched using either the UniGene accession number identified using LocusLink (preferred if available) or can be BLAST searched using the SNP target sequence of interest in FASTA format.

5. SNP Consortium Database

In some embodiments, masked sequences are used to search the SNP Consortium (TSC) database (available at SNP Consortium Ltd public web site). In some embodiments, SNP Consortium searches are conducted concurrently with dbSNP, LocusLink, UniGene, and OMIM searches. The SNP Consortium database includes mapping and allele frequency information. The database is searched via BLAST using the masked input target sequence. The output from the SNP Consortium database includes a TSC accession number and a Goldenpath Contig accession number in addition to mapping and allele frequency information (if known).

6. Genome Databases

In some embodiments, target sequences are used to search genome databases (e.g., including but not limited to the Golden Path Database at University of California at Santa Cruz (UCSC) and GenBank). The GoldenPath database is searched via BLAST using the sequence in FASTA format or using the RS# obtained from dbSNP. GenBank is searched via BLAST using the masked sequence in FASTA format. In some embodiments, GoldenPath and GenBank searches are performed concurrently with TSC and dbSNP searches. In some embodiments, the searches result in the identification of the corresponding gene. Output from GenBank includes a GenBank accession number. Output from both databases includes contig accession numbers.

In some embodiments, a match to an incomplete gene is identified. In these cases, the automated system of the present invention directs the search of databases of unfinished genomic sequences (e.g., including but not limited to The High Throughput Genomic (HTG) Sequences database, a database that includes unfinished sequences from DDBJ, EMBL, and GenBank). Unfinished HTG sequences containing contigs greater than 2 kb are assigned an accession number and deposited in the HTG division. A typical HTG record might consist of all the first pass sequence data generated from a single cosmid, BAC, YAC, or P1 clone that together comprise more than 2 kb and contain one or more gaps. A single accession number is assigned to this collection of sequences and each record includes a clear indication of the status (phase 1 or 2) plus a prominent warning that the sequence data is “unfinished” and may contain errors. The accession number does not change as sequence records are updated; only the most recent version of a HTG record remains in GenBank. ‘Finished’ HTG sequences (phase 3) retain the same accession number, but are moved into the relevant primary GenBank division.

If a gene is identified using an unfinished sequence database, the information is transferred to the Oracle database of the present invention. If a gene is not identified, the automated system periodically (e.g., weekly) searches the databases for such information.

7. Private Databases

In some embodiments of the present invention, private databases are searched. For example, the present invention provides systems and methods for gathering, organizing, and storing sequence information (See e.g., Sections III, IV and V, below). Information obtained by the methods of the present invention may be searched during target sequence analysis to assist in the confirmation or selection of target sequences that are likely to be successful in the desired detection assay (e.g., information obtained from previously successful assays is used to select or predict successful sequences for subsequent assays on the same or similar targets using the same or similar types of detection assay).

8. Patent Databases

In some embodiments of the present invention, patent databases are searched. In some embodiments, a search is conducted to identify patents and patent applications related to a target or probe sequence. For example, patent claims may relate to target sequences, target SNPs, probe sequences and methods of using these compositions. Searchable databases of patented sequences may be public or private. Examples of tools for searching for patented sequences include GENESEQ and The Patent Agent. GENESEQ (Derwent Information, Alexandria Va.) searches for patented sequences in basic patents from 40 patent issuing authorities worldwide. GENESEQ provides a flat file (ASCII) EMBL-based format to enable integration into bioinformatics systems. The Patent Agent (DoubleTwist, Inc., Oakland, Calif.) uses the BLAST2N and BLAST2P algorithms to search Derwent's GENESEQ patent database and GenBank's patent division for sequence patent records matching an input (query) sequence.

E. Processing of Database Information

The collection of information obtained from the database searches is analyzed and/or stored. In some embodiments, the candidate target sequence is identified as a “high probability” target sequences and the results are reported to a user (to recommend production or use) or the target is directly sent on for production (Section III, below) or used. A high probability target sequence is one where the target sequence was confirmed to exist in one or more sequence databases, where there is no identified disagreement between the sequence databases (e.g., disagreement relating to the sequence of the target, the location of the target, or the presence of known mutations within the target region), where the target sequence represents a unique sequence in the samples that are to be assayed, and where the sequence corresponding to the target is considered reliable (i.e., confirmed or completed) sequence. In some embodiments, where a report is sent to a user, the report may include results of each search, a summary of the results, a general indication that the target sequence is a high probability sequences, and/or any other detailed information identified by the searches (e.g., disease association information).

In some embodiments of the present invention, where one or more problems are identified with the candidate target sequence, a report is sent to a user (e.g., the person who input or requested the candidate target sequence or a technician utilizing the systems and methods of the present invention) highlighting the one or more problems. Problems include the presence of repeat or artifact sequences in the candidate target sequences, multiple copies of the target sequence in the sample to be assayed (e.g., in the human genome), absence of the sequence in one or more of the databases, inconsistent results from one or more the databases (e.g., inconsistency as to the sequence corresponding to the target, the location of the target within a genome, the presence or location of a mutation or SNP to be assayed, and the presence or absence of one or more additional mutations or SNPs within the target region), and/or the sequence quality (reliability) of the sequence from the databases. In some embodiments, a reliability score is generated based on the presence or absence of one or more of the above potential problems. The reliability score may be sent to the user, or may be used as a signal to cause a further action, such as to begin production and/or to cancel the candidate target sequence.

In some embodiments, the user is given the option to select another target sequence or to proceed with the present target sequence (e.g., to proceed to production). In some embodiments, when problems are identified, the systems of the present invention automatically select and test additional candidate target sequences based on the original requested candidate target sequence (e.g., select neighboring sequences and/or remove problem portions of the sequence). If more reliable sequences are identified, these suggested alternate target sequences are reported to the user.

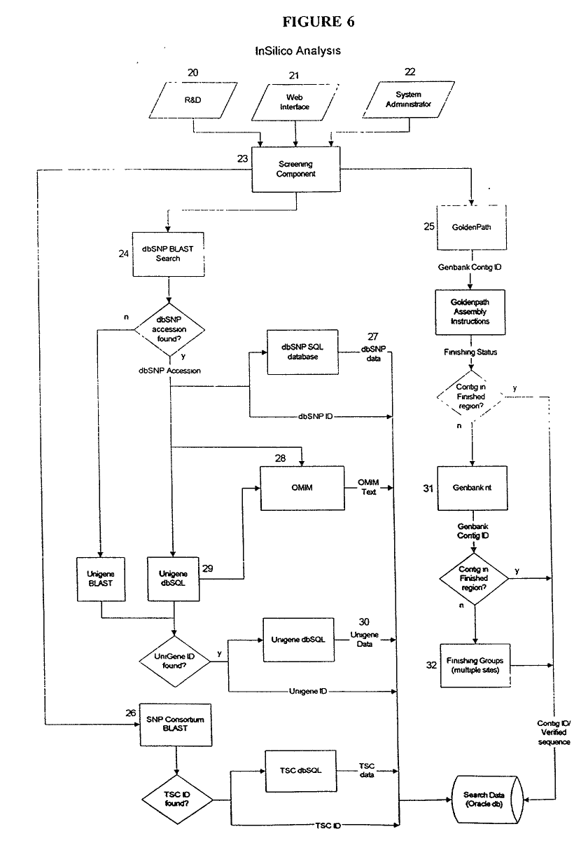

An overview of in silico analysis in some preferred embodiments of the present invention is shown in FIG. 6. The three top boxes represent exemplary sources of target sequences: research & development (e.g., direct input by research personnel) (20), Web interface (sequence input through a communication network) (21), and system administrators (e.g., to test the systems and methods of the present invention) (22). The target sequences are then analyzed by a screening component (23) that masks repeat and artifact sequences. If sequences are suitable for further analysis, they are passed to a series of databases. In the example shown in FIG. 6, the sequences are simultaneously sent to dbSNP (24), GoldenPath (25), and SNP Consortium (26) databases. If a dbSNP accession number is available, dbSNP data (27) is collected and stored and the dbSNP accession number is used to search the Unigene database (29). The dbSNP accession number may also be used to search the OMIM database (28) (which may also be searched after any other database search). If a dbSNP accession is not identified, the target sequence information is passed to the Unigene database (29). If a Unigene identification is found, Unigene data (30) is collected and stored.

The target sequence information sent to the GoldenPath database (25) is used to identify a GoldenPath identification number and to check the reliability status of the sequence. If the sequence is considered “finished” sequence, GoldenPath data is collected and stored. If the sequence is not finished, the GenBank database (31) is searched to identify a GenBank contig identification number and to determine if the contig is considered “finished.” If the contig is finished, data is collected and stored. If the contig is not considered finished, a request for additional sequence data is placed with the group responsible with finishing the sequence of the region (32). If sequence data is available, data from the finishing group is collected and stored.

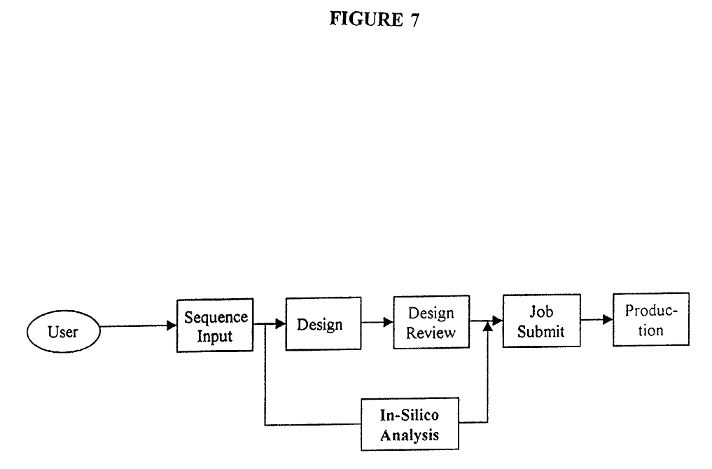

The target sequence information sent to the SNP Consortium database (26) is used to identify a TSC identification number and TSC data, if available, is collected and stored. In some embodiments, one or more database accession numbers (e.g., LocusLink accession number) are provided during the original target sequence input or at any time thereafter, and said accession numbers are used to direct searches in the corresponding database (e.g., LocusLink database) or other databases. To the extent that databases searches are conducted solely to obtain an accession number for use in searching other databases, pre-entry of the accession number reduced the time required for in silico analysis. All of the collected data is stored in a database and used to generate reports and/or reliability scores for use in determining whether production of an assay directed at the target sequence should proceed. In some embodiments, if production is to proceed, information from the in silico analysis and design analysis (Section II, below) is sent to a production facility. The flow of information from sequence input to production in some embodiments of the present invention is shown FIG. 7.

II. Detection Assay Design

There are a wide variety of detection technologies available for determining the sequence of a target nucleic acid at one or more locations. For example, there are numerous technologies available for detecting the presence or absence of SNPs. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid. A number of these technologies are described in detail, in Section IV, below.

The present invention provides systems and methods for the design of oligonucleotides for use in detection assays. In particular, the present invention provides systems and methods for the design of oligonucleotides that successfully hybridize to appropriate regions of target nucleic acids (e.g., regions of target nucleic acids that do not contain secondary structure) under the desired reaction conditions (e.g., temperature, buffer conditions, etc.) for the detection assay. The systems and methods also allow for the design of multiple different oligonucleotides (e.g., oligonucleotides that hybridize to different portions of a target nucleic acid or that hybridize to two or more different target nucleic acids) that all function in the detection assay under the same or substantially the same reaction conditions. These systems and methods may also be used to design control samples that work under the experimental reaction conditions.

While the systems and methods of the present invention are not limited to any particular detection assay, the following description illustrates the invention when used in conjunction with the INVADER assay (Third Wave Technologies, Madison Wis.; See e.g., U.S. Pat. Nos. 5,846,717, 5,985,557, 5,994,069, and 6,001,567 and PCT Publications WO 97/27214 and WO 98/42873, incorporated herein by reference in their entireties) to detect a SNP. The INVADER assay provides ease-of-use and sensitivity levels that, when used in conjunction with the systems and methods of the present invention, find use in detection panels, ASRs, and clinical diagnostics. One skilled in the art will appreciate that specific and general features of this illustrative example are generally applicable to other detection assays.

A. INVADER Assay

The INVADER assay provides means for forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

The INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

B. Oligonucleotide Design for the INVADER Assay

In some embodiments where an oligonucleotide is designed for use in the INVADER assay to detect a SNP, the sequence(s) of interest are entered into the INVADERCREATOR program (Third Wave Technologies, Madison, Wis.). As described above, sequences may be input for analysis from any number of sources, either directly into the computer hosting the INVADERCREATOR program, or via a remote computer linked through a communication network (e.g., a LAN, Intranet or Internet network). The program designs probes for both the sense and antisense strand. Strand selection is generally based upon the ease of synthesis, minimization of secondary structure formation, and manufacturability. In some embodiments, the user chooses the strand for sequences to be designed for. In other embodiments, the software automatically selects the strand. By incorporating thermodynamic parameters for optimum probe cycling and signal generation (Allawi and SantaLucia, Biochemistry, 36:10581 [1997]), oligonucleotide probes may be designed to operate at a pre-selected assay temperature (e.g., 63° C.). Based on these criteria, a final probe set (e.g., primary probes for 2 alleles and an INVADER oligonucleotide) is selected.

In some embodiments, the INVADERCREATOR system is a web-based program with secure site access that contains a link to BLAST (available at the National Center for Biotechnology Information, National Library of Medicine, National Institutes of Health website) and that can be linked to RNAstructure (Mathews et al., RNA 5:1458 [1999]), a software program that incorporates mfold (Zuker, Science, 244:48 [1989]). RNAstructure tests the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system was configured with Oracle to work well with UNIX systems, as most genome centers are UNIX-based.

In some embodiments, the INVADERCREATOR analysis is provided on a separate server (e.g., a Sun server) so it can handle analysis of large batch jobs. For example, a customer can submit up to 2,000 SNP sequences in one email. The server passes the batch of sequences on to the INVADERCREATOR software, and, when initiated, the program designs detection assay oligonucleotide sets. In some embodiments, probe set designs are returned to the user within 24 hours of receipt of the sequences.

Each INVADER reaction includes at least two target sequence-specific, unlabeled oligonucleotides for the primary reaction: an upstream INVADER oligonucleotide and a downstream Probe oligonucleotide. The INVADER oligonucleotide is generally designed to bind stably at the reaction temperature, while the probe is designed to freely associate and disassociate with the target strand, with cleavage occurring only when an uncut probe hybridizes adjacent to an overlapping INVADER oligonucleotide. In some embodiments, the probe includes a 5′ flap or “arm” that is not complementary to the target, and this flap is released from the probe when cleavage occurs. In some embodiments, the released flap participates as an INVADER oligonucleotide in a secondary reaction.

The following discussion provides one example of how a user interface for an INVADERCREATOR program may be configured.

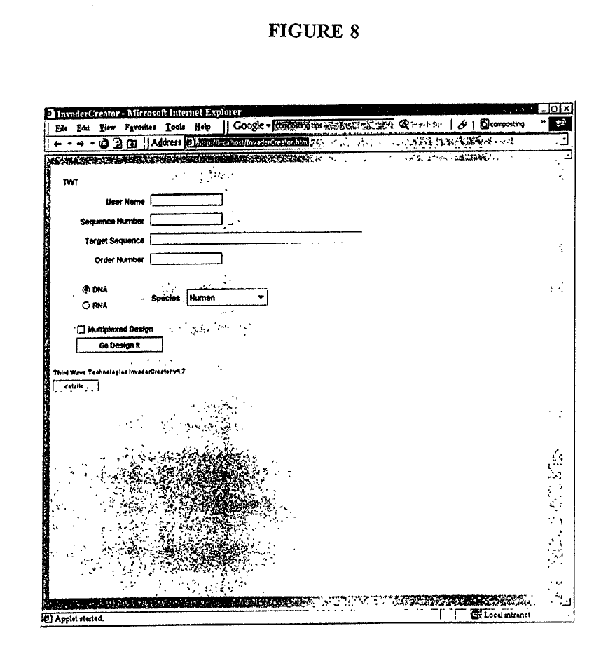

The user opens a work screen (FIG. 8), e.g., by clicking on an icon on a desktop display of a computer (e.g., a Windows desktop). The user enters information related to the target sequence for which an assay is to be designed. In some embodiments, the user enters a target sequence. In other embodiments, the user enters a code or number that causes retrieval of a sequence from a database. In still other embodiments, additional information may be provided, such as the user's name, an identifying number associated with a target sequence, and/or an order number. In preferred embodiments, the user indicates (e.g. via a check box or drop down menu) that the target nucleic acid is DNA or RNA. In other preferred embodiments, the user indicates the species from which the nucleic acid is derived. In particularly preferred embodiments, the user indicates whether the design is for monoplex (i.e., one target sequence or allele per reaction) or multiplex (i.e., multiple target sequences or alleles per reaction) detection. When the requisite choices and entries are complete, the user starts the analysis process. In one embodiment, the user clicks a “Go Design It” button to continue.

In some embodiments, the software validates the field entries before proceeding. In some embodiments, the software verifies that any required fields are completed with the appropriate type of information. In other embodiments, the software verifies that the input sequence meets selected requirements (e.g., minimum or maximum length, DNA or RNA content). If entries in any field are not found to be valid, an error message or dialog box may appear. In preferred embodiments, the error message indicates which field is incomplete and/or incorrect. Once a sequence entry is verified, the software proceeds with the assay design.

In some embodiments, the information supplied in the order entry fields specifies what type of design will be created. In preferred embodiments, the target sequence and multiplex check box specify which type of design to create. Design options include but are not limited to SNP assay, Multiplexed SNP assay (e.g., wherein probe sets for different alleles are to be combined in a single reaction), Multiple SNP assay (e.g., wherein an input sequence has multiple sites of variation for which probe sets are to be designed), and Multiple Probe Arm assays.

In some embodiments, the INVADERCREATOR software is started via a Web Order Entry (WebOE) process (i.e., through an Intra/Internet browser interface) and these parameters are transferred from the WebOE via applet <param> tags, rather than entered through menus or check boxes.

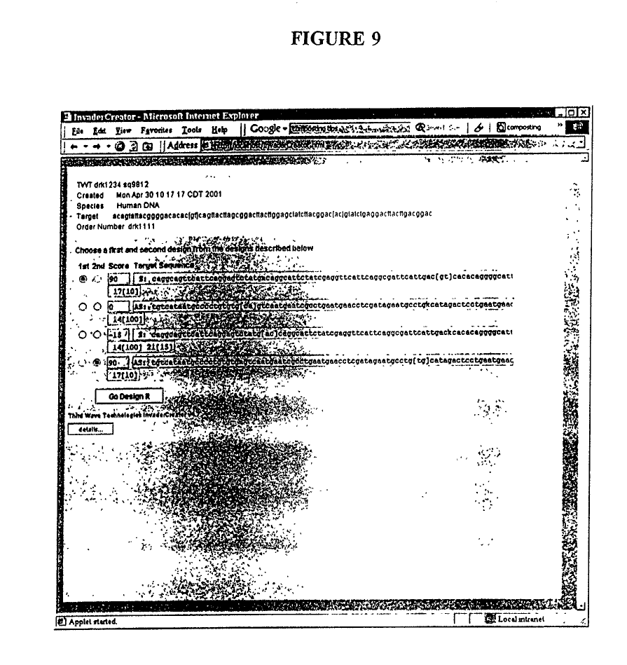

In the case of Multiple SNP Designs, the user chooses two or more designs to work with. In some embodiments, this selection opens a new screen view (e.g., a Multiple SNP Design Selection view FIG. 9). In some embodiments, the software creates designs for each locus in the target sequence, scoring each, and presents them to the user in this screen view. The user can then choose any two designs to work with. In some embodiments, the user chooses a first and second design (e.g., via a menu or buttons) and clicks a “Go Design It” button to continue.

To select a probe sequence that will perform optimally at a pre-selected reaction temperature, the melting temperature (Tm) of the SNP to be detected is calculated using the nearest-neighbor model and published parameters for DNA duplex formation (Allawi and SantaLucia, Biochemistry, 36:10581 [1997]). In embodiments wherein the target strand is RNA, parameters appropriate for RNA/DNA heteroduplex formation may be used. Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tm to determine the optimal temperature at which to perform a reaction. One way of compensating for these factors is to vary the value provided for the salt concentration within the melting temperature calculations. This adjustment is termed a ‘salt correction’. As used herein, the term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 0.5 M NaCl (SantaLucia, Proc Natl Acad Sci U S A, 95:1460 [1998]) and strand concentrations of about 1 mM of the probe and 1 fM target, the algorithm for used for calculating probe-target melting temperature has been adapted for use in predicting optimal INVADER assay reaction temperature. For a set of 30 probes, the average deviation between optimal assay temperatures calculated by this method and those experimentally determined is about 1.5° C.

The length of the downstream probe to a given SNP is defined by the temperature selected for running the reaction (e.g., 63° C.). Starting from the position of the variant nucleotide on the target DNA (the target base that is paired to the probe nucleotide 5′ of the intended cleavage site), and adding on the 3′ end, an iterative procedure is used by which the length of the target-binding region of the probe is increased by one base pair at a time until a calculated optimal reaction temperature (Tₘ plus salt correction to compensate for enzyme effect) matching the desired reaction temperature is reached. The non-complementary arm of the probe is preferably selected to allow the secondary reaction to cycle at the same reaction temperature. The entire probe oligonucleotide is screened using programs such as mfold (Zuker, Science, 244: 48 [1989]) or Oligo 5.0 (Rychlik and Rhoads, Nucleic Acids Res, 17: 8543 [1989]) for the possible formation of dimer complexes or secondary structures that could interfere with the reaction. The same principles are also followed for INVADER oligonucleotide design. Briefly, starting from the position N on the target DNA, the 3′ end of the INVADER oligonucleotide is designed to have a nucleotide not complementary to either allele suspected of being contained in the sample to be tested. The mismatch does not adversely affect cleavage (Lyamichev et al., Nature Biotechnology, 17: 292 [1999]), and it can enhance probe cycling, presumably by minimizing coaxial stabilization effects between the two probes. Additional residues complementary to the target DNA starting from residue N−1 are then added in the 5′ direction until the stability of the INVADER oligonucleotide-target hybrid exceeds that of the probe (and therefore the planned assay reaction temperature), generally by 15-20° C.

It is one aspect of the assay design that the all of the probe sequences may be selected to allow the primary and secondary reactions to occur at the same optimal temperature, so that the reaction steps can run simultaneously. In an alternative embodiment, the probes may be designed to operate at different optimal temperatures, so that the reaction steps are not simultaneously at their temperature optima.

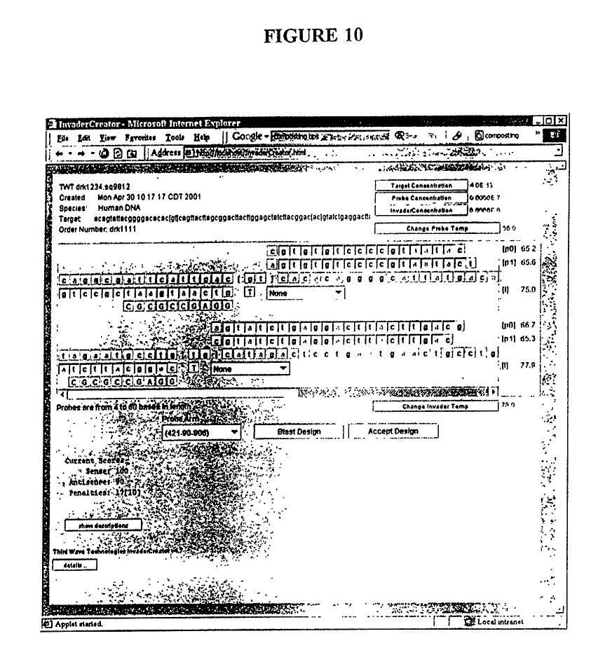

In some embodiments, the software provides the user an opportunity to change various aspects of the design including but not limited to: probe, target and INVADER oligonucleotide temperature optima and concentrations; blocking groups; probe arms; dyes, capping groups and other adducts; individual bases of the probes and targets (e.g., adding or deleting bases from the end of targets and/or probes, or changing internal bases in the INVADER and/or probe and/or target oligonucleotides). In some embodiments, changes are made by selection from a menu. In other embodiments, changes are entered into text or dialog boxes. In preferred embodiments, this option opens a new screen (e.g., a Designer Worksheet view, FIG. 10).

In some embodiments, the software provides a scoring system to indicate the quality (e.g., the likelihood of performance) of the assay designs. In one embodiment, the scoring system includes a starting score of points (e.g., 100 points) wherein the starting score is indicative of an ideal design, and wherein design features known or suspected to have an adverse affect on assay performance are assigned penalty values. Penalty values may vary depending on assay parameters other than the sequences, including but not limited to the type of assay for which the design is intended (e.g., monoplex, multiplex) and the temperature at which the assay reaction will be performed. The following example provides an illustrative scoring criteria for use with some embodiments of the INVADER assay based on an intelligence defined by experimentation. Examples of design features that may incur score penalties include but are not limited to the following [penalty values are indicated in brackets, first number is for lower temperature assays (e.g., 62-64° C.), second is for higher temperature assays (e.g., 65-66° C.)]:

1. [100:100] 3′ end of INVADER oligonucleotide
resembles the probe arm:

ARM SEQUENCE:
Arm 1: CGCGCCGAGG
Arm 2: ATGACGTGGCAGAC
Arm 3: ACGGACGCGGAG
Arm 4: TCCGCGCGTCC

PENALTY AWARDED IF INVADER ENDS IN:
5′ . . . GAGGX or 5′ . . . GAGGXX
5′ . . . CAGACX or 5′ . . . CAGACXX
5′ . . . GGAGX or 5′ . . . GGAGXX
5′ . . . GTCCX or 5′ . . . GTCCXX

2. [70:70] a probe has 5-base stretch (i.e., 5 of the same base in a row) containing the polymorphism;

3. [60:60] a probe has 5-base stretch adjacent to the polymorphism;

4. [50:50] a probe has 5-base stretch one base from the polymorphism;

5. [40:40] a probe has 5-base stretch two bases from the polymorphism;

6. [50:50] probe 5-base stretch is of Gs—additional penalty;

7. [100: 100] a probe has 6-base stretch anywhere;

8. [90:90] a two or three base sequence repeats at least four times;

9. [100:100] a degenerate base occurs in a probe;

10. [60:90] probe hybridizing region is short (13 bases or less for designs 65-67° C.; 12 bases or less for designs 62-64° C.)

11. [40:90] probe hybridizing region is long (29 bases or more for designs 65-67° C., 28 bases or more for designs 62-64° C.)

12. [5:5] probe hybridizing region length—per base additional penalty

13. [80:80] Ins/Del design with poor discrimination in first 3 bases after probe arm

14. [100:100] calculated INVADER oligonucleotide Tm within 7.5° C. of probe target Tm (designs 65-67° C. with INVADER oligonucleotide less than <70.5° C., designs 62-64° C. with INVADER oligonucleotide <69.5° C.

15. [20:20] calculated probes Tms differ by more than 2.0° C.

16. [100:100] a probe has calculated Tm 2° C. less than its target Tm

17. [10:10] target of one strand 8 bases longer than that of other strand

18. [30:30] INVADER oligonucleotide has 6-base stretch anywhere—initial penalty

19. [70:70] INVADER oligonucleotide 6-base stretch is of Gs—additional penalty

20. [15:15] probe hybridizing region is 14, 15 or 24-28 bases long (65-67° C.) or 13,14 or 26,27 bases long (62-64° C.)

21. [15:15] a probe has a 4-base stretch of Gs containing the polymorphism

In particularly preferred embodiments, temperatures for each of the oligonucleotides in the designs are recomputed and scores are recomputed as changes are made. In some embodiments, score descriptions can be seen by clicking a “descriptions” button. In some embodiments, a BLAST search option is provided. In preferred embodiments, a BLAST search is done by clicking a “BLAST Design” button. In some embodiments, this action brings up a dialog box describing the BLAST process. In preferred embodiments, the BLAST search results are displayed as a highlighted design on a Designer Worksheet.

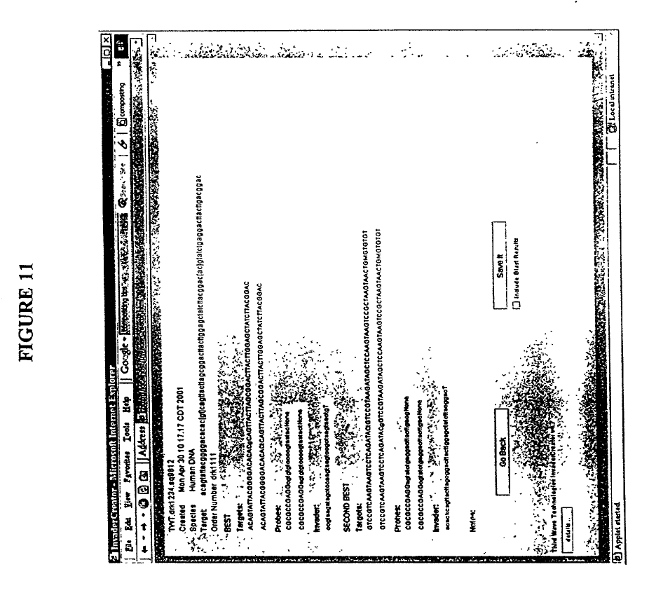

In some embodiments, a user accepts a design by clicking an “Accept” button. In other embodiments, the program approves a design without user intervention. In preferred embodiments, the program sends the approved design to a next process step (e.g., into production; into a file or database). In some embodiments, the program provides a screen view (e.g., an Output Page, FIG. 11), allowing review of the final designs created and allowing notes to be attached to the design. In preferred embodiments, the user can return to the Designer Worksheet (e.g., by clicking a “Go Back” button) or can save the design (e.g., by clicking a “Save It” button) and continue (e.g., to submit the designed oligonucleotides for production).

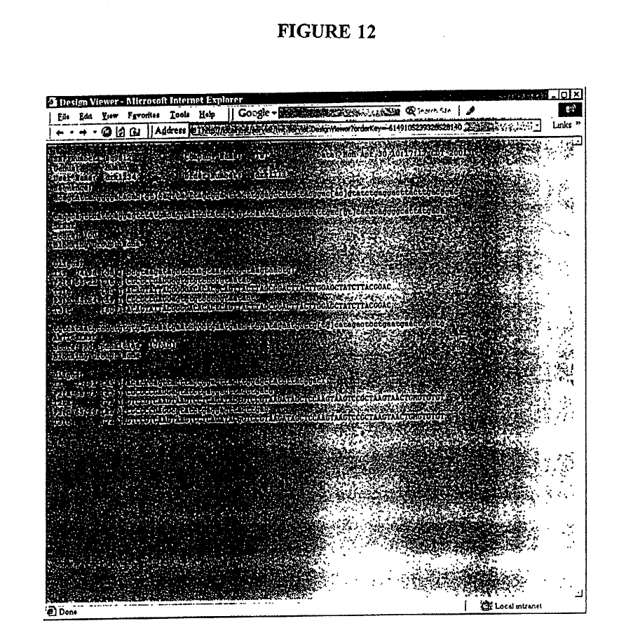

In some embodiments, the program provides an option to create a screen view of a design optimized for printing (e.g., a text-only view) or other export (e.g., an Output view, FIG. 12). In preferred embodiments, the Output view provides a description of the design particularly suitable for printing, or for exporting into another application (e.g., by copying and pasting into another application). In particularly preferred embodiments, the Output view opens in a separate window.

The present invention is not limited to the use of the INVADERCREATOR software. Indeed, a variety of software programs are contemplated and are commercially available, including, but not limited to GCG Wisconsin Package (Genetics computer Group, Madison, Wis.) and Vector NTI (Informax, Rockville, Md.).

III. Detection Assay Production

The present invention provides a high-throughput detection assay production system, allowing for high-speed, efficient production of thousands of detection assays. The high-throughput production systems and methods allow sufficient production capacity to facilitate full implementation of the funnel process described above—allowing comprehensive of all known (and newly identified) markers.

In some embodiments of the present invention, oligonucleotides and/or other detection assay components (e.g., those designed by the INVADERCREATOR software and directed to target sequences analyzed by the in silico systems and methods) are synthesized. In preferred embodiments, oligonucleotide synthesis is performed in an automated and coordinated manner. As discussed in more detail below, in some embodiments, produced detection assay are tested against a plurality of samples representing two or more different individuals or alleles (e.g., samples containing sequences from individuals with different ethnic backgrounds, disease states, etc.) to demonstrate the viability of the assay with different individuals.

In some embodiments, the present invention provides an automated DNA production process. In some embodiments, the automated DNA production process includes an oligonucleotide synthesizer component and an oligonucleotide processing component. In some embodiments, the oligonucleotide production component includes multiple components, including but not limited to, an oligonucleotide cleavage and deprotection component, an oligonucleotide purification component, an oligonucleotide dry down component; an oligonucleotide de-salting component, an oligonucleotide dilute and fill component, and a quality control component. In some embodiments, the automated DNA production process of the present invention further includes automated design software and supporting computer terminals and connections, a product tracking system (e.g., a bar code system), and a centralized packaging component. In some embodiments, the components are combined in an integrated, centrally controlled, automated production system. The present invention thus provides methods of synthesizing several related oligonucleotides (e.g., components of a kit) in a coordinated manner. The automated production systems of the present invention allow large scale automated production of detection assays for numerous different target sequences.

A. Oligonucleotide Synthesis Component

Once a particular oligonucleotide sequence or set of sequences has been chosen, sequences are sent (e.g., electronically) to a high-throughput oligonucleotide synthesizer component. In some preferred embodiments, the high-throughput synthesizer component contains multiple DNA synthesizers.

In some embodiments, the synthesizers are arranged in banks. For example, a given bank of synthesizers may be used to produce one set of oligonucleotides (e.g., for an INVADER or PCR reaction). The present invention is not limited to any one synthesizer. Indeed, a variety of synthesizers are contemplated, including, but not limited to MOSS EXPEDITE 16-channel DNA synthesizers (PE Biosystems, Foster City, Calif.), OligoPilot (Amersham Pharmacia,), the 3900 and 3948 48-Channel DNA synthesizers (PE Biosystems, Foster City, Calif.), and the high-throughput synthesizer described in PCT Publication WO 01/41918. In some embodiments, synthesizers are modified or are wholly fabricated to meet physical or performance specifications particularly preferred for use in the synthesis component of the present invention. In some embodiments, two or more different DNA synthesizers are combined in one bank in order to optimize the quantities of different oligonucleotides needed. This allows for the rapid synthesis (e.g., in less than 4 hours) of an entire set of oligonucleotides (all the oligonucleotide components needed for a particular assay, e.g., for detection of one SNP using an INVADER assay).

In some embodiments the DNA synthesizer component includes at least 100 synthesizers. In other embodiments, the DNA synthesizer component includes at least 200 synthesizers. In still other embodiments, the DNA synthesizer component includes at least 250 synthesizers. In some embodiments, the DNA synthesizers are run 24 hours a day.

1. Automated Reagent Supply

In some embodiments, the DNA synthesizers in the oligonucleotide synthesis component further comprise an automated reagent supply system. The automated reagent supply system delivers reagents necessary for synthesis to the synthesizers from a central supply area. For example, in some embodiments, acetonitrile is supplied via tubing (e.g., stainless steel tubing) through the automated supply system. De-blocking solution may also be supplied directly to DNA synthesizers through tubing. In some preferred embodiments, the reagent supply system tubing is designed to connect directly to the DNA synthesizers without modifying the synthesizers. Additionally, in some embodiments, the central reagent supply is designed to deliver reagents at a constant and controlled pressure. The amount of reagent circulating in the central supply loop is maintained at 8 to 12 times the level needed for synthesis in order to allow standardized pressure at each instrument. The excess reagent also allows new reagent to be added to the system without shutting down. In addition, the excess of reagent allows different types of pressurized reagent containers to be attached to one system. The excess of reagents in one centralized system further allows for one central system for chemical spills and fire suppression.

In some embodiments, the DNA synthesis component includes a centralized argon delivery system. The system includes high-pressure argon tanks adjacent to each bank of synthesizers. These tanks are connected to large, main argon tanks for backup. In some embodiments, the main tanks are run in series. In other embodiments, the main tanks are set up in banks. In some embodiments, the system further includes an automated tank switching system. In some preferred embodiments, the argon delivery system further comprises a tertiary backup system to provide argon in the case of failure of the primary and backup systems.

In some embodiments, one or more branched delivery components are used between the reagent tanks and the individual synthesizers or banks of synthesizers. For example, in some embodiments, acetonitrile is delivered through a branched metal structure. Where more than one branched delivery component is used, in preferred embodiments, each branched delivery component is individually pressurized.

The present invention is not limited by the number of branches in the branched delivery component. In preferred embodiments, each branched delivery component contains ten or more branches. Reagent tanks may be connected to the branched delivery components using any number of configurations. For example, in some embodiments, a single reagent tank is matched with a single branched component. In other embodiments, a plurality of reagent tanks is used to supply reagents to one or more branched components. In some such embodiments, the plurality of tanks may be attached to the branched components through a single feed line, wherein one or a subset of the tanks feeds the branched components until empty (or substantially empty), whereby a second tank or subset of tanks is accessed to maintain a continuous supply of reagent to the one or more branched components. To automate the monitoring and switching of tanks, an ultrasonic level sensor may be applied.

In some embodiments, each branch of the branched delivery component provides reagent to one synthesizer or to a bank of synthesizers through connecting tubing. In preferred embodiments, tubing is continuous (i.e., provides a direct connection between the delivery branch and the synthesizer). In some preferred embodiments, the tubing comprises an interior diameter of 0.25 inches or less (e.g., 0.125 inches). In some embodiments, each branch contains one or more valves (preferably one). While the valve may be located at any position along the delivery line, in preferred embodiments, the valve is located in close proximity to the synthesizer. In other embodiments, reagent is provided directly to synthesizers without any joints or valves between the branched delivery component and the synthesizers.

In some embodiments, the solvent is contained in a cabinet designed for the safe storage of flammable chemicals (a “flammables cabinet”) and the branched structure is located outside of the cabinet and is fed by the solvent container through a tube passed through the wall of the cabinet. In other embodiments, the reagent and branched system is stored in an explosion proof room or chamber and the solvent is pumped via tubing through the wall of the explosion proof room. In preferred embodiments, all of the tubing from each of the branches is fed through the wall in at a single location (e.g., through a single hole in the wall).

The reagent delivery system of the present invention provides several advantages. For example, such a system allows each synthesizer to be turned off (e.g., for servicing) independent of the other synthesizers. Use of continuous tubing reduces the number of joints and couplings, the areas most vulnerable to failure, between the reagent sources and the synthesizers, thereby reducing the potential for leakage or blockage in the system. Use of continuous tubing through inaccessible or difficult-to-access areas reduces the likelihood that repairs or service will be needed in such areas. In addition, fewer valves results in cost savings.

In some embodiments, the branched tubing structure further provides a sight glass. In preferred embodiments, the sight glass is located at the top of the branched delivery structure. The sight glass provides the opportunity for visual and physical sampling of the reagent. For example, in some embodiments, the sight glass includes a sampling valve (e.g., to collect samples for quality control). In some embodiments, the site glass serves as a trap for gas bubbles, to prevent bubbles from entering the connecting tubing. In other embodiments, the sight glass contains a vent (e.g., a solonoid valve) for de-gassing of the system. In some embodiments, scanning of the sight glass (e.g., spectrophotometrically) and sampling are automated. The automated system provides quality control and feedback (e.g., the presence of contamination).

In other embodiments, the present invention provides a portable reagent delivery system. In some embodiments, the portable reagent delivery system comprises a branched structure connected to solvent tanks that are contained in a flammables cabinet. In preferred embodiments, one reagent delivery system is able to provide sufficient reagent for 40 or more synthesizers. These portable reagent delivery systems of the present invention facilitate the operation of mobile (portable) synthesis facilities. In another embodiment, these portable reagent delivery systems facilitate the operation of flexible synthesis facilities that can be easily re-configured to meet particular needs of individual synthesis projects or contracts. In some embodiments, a synthesis facility comprises multiple portable reagent delivery systems.

2. Waste Collection

In some embodiments, the DNA synthesis component further comprises a centralized waste collection system. The centralized waste collection system comprises cache pots for central waste collection. In some embodiments, the cache pots include level detectors such that when waste level reaches a preset value, a pump is activated to drain the cache into a central collection reservoir. In preferred embodiments, ductwork is provided to gather fumes from cache pots. The fumes are then vented safely through the roof, avoiding exposure of personnel to harmful fumes. In preferred embodiments, the air handling system provides an adequate amount of air exchange per person to ensure that personnel are not exposed to harmful fumes. The coordinated reagent delivery and waste removal systems increase the safety and health of workers, as well as improving cost savings.

In some embodiments, the solvent waste disposal system comprises a waste transfer system. In some preferred embodiments, the system contains no electronic components. In some preferred embodiments, the system comprises no moving parts. For example, in some embodiments, waste is first collected in a liquid transfer drum designed for the safe storage of flammable waste. In some embodiments, waste is manually poured into the drum through a waste channel. In preferred embodiments, solvent waste is automatically transported (e.g., through tubing) directly from synthesizers to the drum. To drain the liquid transfer drum, argon is pumped from a pressurized gas line into the drum through a first opening, forcing solvent waste out an output channel at a second opening (e.g., through tubing) into a centralized waste collection area. In preferred embodiments, the argon is pumped at low pressure (e.g., 3-10 pounds per square inch (psi), preferably 5 psi or less). In some embodiments, the drum contains a sight glass to visualize the solvent level. In some embodiments, the level is visualized manually and the disposal system is activated when the drum has reached a selected threshold level. In other embodiments, the level is automatically detected and the disposal system is automatically activated when the drum has reached the threshold level.

The solvent waste transfer system of the present invention provides several advantages over manual collection and complex systems. The solvent waste system of the present invention is intrinsically safe, as it can be designed with no moving or electrical parts. For example, the system described above is suitable for use in Division I/Class I space under EPA regulations.

3. Centralized Control System

In some embodiments, all of the DNA synthesizers in the synthesis component are attached to a centralized control system. The centralized control system controls all areas of operation, including, but not limited to, power, pressure, reagent delivery, waste, and synthesis. In some preferred embodiments, the centralized control system includes a clean electrical grid with uninterrupted power supply. Such a system minimizes power level fluctuations. In additional preferred embodiments, the centralized control system includes alarms for air flow, status of reagents, and status of waste containers. The alarm system can be monitored from the central control panel. The centralized control system allows additions, deletions, or shutdowns of one synthesizer or one block of synthesizers without disrupting operations of other instruments. The centralized power control allows user to turn instruments off instrument by instrument, bank by bank, or the entire module.

B. Oligonucleotide Processing Components

In some embodiments, the automated DNA production process further comprises one or more oligonucleotide production components, including, but not limited to, an oligonucleotide cleavage and deprotection component, an oligonucleotide purification component, a dry-down component, a desalting component, a dilution and fill component, and a quality control component.

1. Oligonucleotide Cleavage and Deprotection

After synthesis is complete, the oligonucleotides are moved to the cleavage and deprotection station. In some embodiments, the transfer of oligonucleotides to this station is automated and controlled by robotic automation. In some embodiments, the entire cleavage and deprotection process is performed by robotic automation. In some embodiments, NH₄OH for deprotection is supplied through the automated reagent supply system.

Accordingly, in some embodiments, oligonucleotide deprotection is performed in multi-sample containers (e.g., 96 well covered dishes) in an oven. This method is designed for the high-throughput system of the present invention and is capable of the simultaneous processing of large numbers of samples. This method provides several advantages over the standard method of deprotection in vials. For example, sample handling is reduced (e.g., labeling of vials dispensing of concentrated NH₄OH to individual vials, as well as the associated capping and uncapping of the vials, is eliminated). This reduces the risks of contamination or mislabeling and decreases processing time. Where such methods are used to replace human pipetting of samples and capping of vials, the methods save many labor hours per day. The method also reduces consumable requirements by eliminating the need for vials and pipette tips, reduces equipment needs by eliminating the need for pipettes, and improves worker safety conditions by reducing worker exposure to ammonium hydroxide. The potential for repetitive motion disorders is also reduced. Deprotection in a multi-well plate further has the advantage that the plate can be directly placed on an automated desalting apparatus (e.g., TECAN Robot).

During the development of the present invention, the plate was optimized to be functional and compatible with the deprotection methods. In some embodiments, the plate is designed to be able to hold as much as two milliliters of oligonucleotide and ammonium hydroxide. If deep well plates are used, automated downstream processing steps may need to be altered to ensure that the full volume of sample is extracted from the wells. In some embodiments, the multi-well plates used in the methods of the present invention comprise a tight sealing lid/cover to protect from evaporation, provide for even heating, and are able to withstand temperatures necessary for deprotection. Attempts with initial plates were not successful, having problems with lids that were not suitably sealed and plates that did not withstand deprotection temperatures.

In some embodiments (e.g., processing of target and INVADER oligonucleotides), oligonucleotides are cleaved from the synthesis support in the multi-well plates. In other embodiments (e.g., processing of probe oligonucleotides), oligonucleotides are first cleaved from the synthesis column and then transferred to the plate for deprotection.

2. Oligonucleotide Purification

In some embodiments, following deprotection and cleavage from the solid support, oligonucleotides are further purified. Any suitable purification method may be employed, including, but not limited to, high pressure liquid chromatography (HPLC) (e.g., using reverse phase C18 and ion exchange), reverse phase cartridge purification, and gel electrophoresis. However, in preferred embodiments, purification is carried out using ion exchange HPLC chromatography.

In some embodiments, multiple HPLC instruments are utilized, and integrated into banks (e.g., banks of 8 HPLC instruments). Each bank is referred to as an HPLC module. Each HPLC module consists of an automated injector (e.g., including, but not limited to, Leap Technologies 8-port injector) connected to each bank of automated HPLC instruments (e.g., including, but not limited to, Beckman-Coulter HPLC instruments). The automatic Leap injector can handle four 96-well plates of cleaved and deprotected oligonucleotides at a time. The Leap injector automatically loads a sample onto each of the HPLCs in a given bank. The use of one injector with each bank of HPLC provides the advantage of reducing labor and allowing integrated processing of information.

In some embodiments, oligonucleotides are purified on an ion exchange column using a salt gradient. Any suitable ion exchange functionality or support may be utilized, including but not limited to, Source 15 Q ion exchange resin (Pharmacia). Any suitable salt may be utilized for elution of oligonucleotides from the ion exchange column, including but not limited to, sodium chloride, acetonitrile, and sodium perchlorate. However, in preferred embodiments, a gradient of sodium perchlorate in acetonitrile and sodium acetate is utilized.

In some embodiments, the gradient is run for a sufficient time course to capture a broad range of sizes of oligonucleotides. For example, in some embodiments, the gradient is a 54 minute gradient carried out using the method described in Tables 1 and 2. Table 1 describes the HPLC protocol for the gradient. The time column represents the time of the operation. The module column represents the equipment that controls the operation. The function column represents the function that the HPLC is performing. The value column represents the value of the HPLC function at the time specified in the time column. Table 2 describes the gradient used in HPLC purification. The column temperature is 65° C. Buffer A is 20 mM Sodium Perchlorate, 20 mM Sodium Acetate, 10 Acetonitrile, pH 7.35. Buffer B is 600 mM Sodium Perchlorate, 20 mM Sodium Acetate, 10 Acetonitrile, pH 7.35.

In some embodiments, the gradient is shortened. In preferred embodiments, the gradient is shortened so that a particular gradient range suitable for the elution of a particular oligonucleotide being purified is accomplished in a reduced amount of time. In other preferred embodiments, the gradient is shortened so that a particular gradient range suitable for the elution of any oligonucleotide having a size within a selected size range is accomplished in a reduced amount of time. This latter embodiment provides the advantages that the worker performing HPLC need not have foreknowledge of the size of an oligonucleotide within the selected size range, and the protocol need not be altered for purification of any oligonucleotide having a size within the range.

In a particularly preferred embodiment, the gradient is a 34 minute gradient described in the Tables 3 and 4. The parameters and buffer compositions are as described for Tables 1 and 2 above. Reducing the gradient to 34 minutes increases the capacity of synthesis per HPLC instrument and reduces buffer usage by 50% compared to the 54 minute protocol described above. The 34 minute HPLC method of the present invention has the further advantage of being optimized to be able to separate oligonucleotides of a length range of 23-39 nucleotides without any changes in the protocol for the different lengths within the range. Previous methods required changes for every 2-3 nucleotide change in length. In yet other embodiments, the gradient time is reduced even further (e.g., to less than 30 minutes, preferably to less than 20 minutes, and even more preferably, to less than 15 minutes). Any suitable method may be utilized that meets the requirements of the present invention (e.g., able to purify a wide range of oligonucleotide lengths using the same protocol).

In some embodiments, separate sets of HPLC conditions, each selected to purify oligonucleotides within a different size range, may be provided (e.g., may be run on separate HPLCs or banks of HPLCs). Thus, in some embodiments of the present invention, a first bank of HPLCs are configured to purify oligonucleotides using a first set of purification conditions (e.g., for 23-39 mers), while second and third banks are used for the shorter and longer oligonucleotides. Use of this system allows for automated purification without the need to change any parameters from purification to purification and decreases the time required for oligonucleotide production.

In some embodiments, the HPLC station is equipped with a central reagent supply system. In some embodiments, the central reagent system includes an automated buffer preparation system. The automated buffer preparation system includes large vat carboys that receive pre-measured reagents and water for centralized buffer preparation. The buffers (e.g., a high salt buffer and a low salt buffer) are piped through a circulation loop directly from the central preparation area to the HPLCs. In some embodiments, the conductivity of the solution in the circulation loop is monitored to verify correct content and adequate mixing. In addition, in some embodiments, circulation lines are fitted with venturis for static mixing of the solutions as they are circulated through the piping loop. In still further embodiments, the circulation lines are fitted with 0.05 μm filters for sterilization.

In some preferred embodiments, the HPLC purification step is carried out in a clean room environment. The clean room includes a HEPA filtration system. All personnel in the clean room are outfitted with protective gloves, hair coverings, and foot coverings.

In preferred embodiments, the automated buffer prep system is located in a non-clean room environment and the prepared buffer is piped through the wall into the clean room.

Each purified oligonucleotide is collected into a tube (e.g., a 50-ml conical tube) in a carrying case in the fraction collector. Collection is based on a set method, which is triggered by an absorbance rate change within a predetermined time window. In some embodiments, the method uses a flow rate of 5 ml/min (the maximum rate of the pumps is 10 ml/min.) and each column is automatically washed before the injector loads the next sample.

TABLE 1
(Det = detector; % B = percent of buffer B; flow rate values in ml/min)
54 Minute HPLC Method

| Time (min) | Module | Function | Value | Duration (min) |
|---|---|---|---|---|
| 0 | Pump | % B | 22.00 | 4.0 |
| 0 | Det 166-3 | Autozero ON | | |
| 0 | Det 166-3 | Relay ON | 3.0 | 0.10 |
| 4 | Pump | % B | 37.00 | 43.00 |
| 47 | Pump | % B | 100.00 | 0.50 |
| 47.5 | Pump | Flow Rate | 7.5 | 0.00 |
| 50.0 | Pump | % B | 5.0 | 0.50 |
| 53.45 | Det 166-3 | Stop Data | | |

TABLE 2
54 Minute HPLC Method

| Time | | Gradient | | Flow Rate |
|---|---|---|---|---|
| 0 | | 5% B/95% | A | 5 ml/min |
| 0-4 | min | 5-22% | B | 5 ml/min |
| 4-47 | min | 22-37% | B | 5 ml/min |
| 47-47.5 | min | 37-100% | B | 7.5 ml/min |
| 47.5-50 | min | 100% | B | 7.5 ml/min |
| 50-50.5 | min | 100-5% | B | 7.5 ml/min |
| 50.5-53.5 | min | 5% | B | 7.5 ml/min |

TABLE 3
34 Minute HPLC Method

| Time (min) | Module | Function | Value | Duration |
|---|---|---|---|---|
| 0 | Pump | % B | 26.00 | 2.0 |
| 0 | Det 166-3 | Autozero ON | | |
| 0 | Det 166-3 | Relay ON | 3.0 | 0.10 |
| 2 | Pump | % B | 36.00 | 27.00 |
| 29 | Pump | % B | 100.00 | 0.50 |
| 29.5 | Pump | Flow Rate | 7.5 | 0.00 |
| 32 | Pump | % B | 5.0 | 0.50 |
| 33.45 | Det 166-3 | Stop Data | | |

TABLE 4
34 Minute HPLC Method

| Time | | Gradient | | Flow Rate |
|---|---|---|---|---|
| 0 | | 5% B/95% | A | 5 ml/min |
| 0-2 | min | 5-26% | B | 5 ml/min |
| 2-29 | min | 26-36% | B | 5 ml/min |
| 29-29.5 | min | 36-100% | B | 6.5 ml/min |
| 29.5-32 | min | 100% | B | 7.5 ml/min |
| 32-32.5 | min | 100-5% | B | 7.5 ml/min |
| 32.5-33.5 | min | 5% | B | 7.5 ml/min |

3. Dry-Down Component

When the fraction collector is full of eluted oligonucleotides, they are transferred (e.g., by automated robotics or by hand) to a drying station. For example, in some embodiments, the samples are transferred to customized racks for Genevac centrifugal evaporator to be dried down. In preferred embodiments, the Genevac evaporator is equipped with racks designed to be used in both the Genevac and the subsequent desalting step. The Genevac evaporator decreases drying time, relative to other commercially available evaporators, by 60%.

4. Desalting Component

In some embodiments, following HPLC, oligonucleotides are desalted. In other embodiments, oligonucleotides are not HPLC purified, but instead proceed directly from deprotection to desalting. In some embodiments, the desalting stations have TECAN robot systems for automated desalting. The system employs a rack that has been designed to fit the TECAN robot and the Genevac centrifugal evaporator without transfer to a different rack or holder. The racks are designed to hold the different sizes of desalting columns, such as the NAP-5 and NAP-10 columns. The TECAN robot loads each oligonucleotide onto an individual NAP-5 or NAP-10 column, supplies the buffer, and collects the eluate. If desired, desalted oligonucleotides may be frozen or dried down at this point.

In some embodiments, following desalting, INVADER and target oligonucleotides are analyzed by mass spectroscopy. For example, in some embodiments, a small sample from the desalted oligonucleotide sample is removed (e.g., by a TECAN robot) and spotted on an analysis plate, which is then placed into a mass spectrometer. The results are analyzed and processed by a software routine. Following the analysis, failed oligonucleotides are automatically reordered, while oligonucleotides that pass the analysis are transported to the next processing step. This preliminary quality control analysis removes failed oligonucleotides earlier in the processing, thus resulting in cost savings and improving cycle times.

5. Oligonucleotide Dilution and Fill Component

In some embodiments, the oligonucleotide production process further includes a dilute and fill module. In some embodiments, each module consists of three automated oligonucleotide dilution and normalization stations. Each station consists of a network-linked computer and an automated robotic system (e.g., including but not limited to Biomek 2000). In one embodiment, the pipetting station is physically integrated with a spectrophotometer to allow machine handling of every step in the process. All manipulations are carried out in a HEPA-filtered environment. Dissolved oligonucleotides are loaded onto the Biomek 2000 deck the sequence files are transferred into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses absorbance and the sequence information to prepare a dilution table for each oligonucleotide. The Biomek employs that dilution table to dilute each oligonucleotide appropriately. The instrument then dispenses oligonucleotides into an appropriate vessel (e.g., 1.5 ml microtubes).

In some preferred embodiments, the automated dilution and fill system is able to dilute different components of a kit (e.g., INVADER and probe oligonucleotides) to different concentrations. In other preferred embodiments, the automated dilution and fill module is able to dilute different components to different concentrations specified by the end user.

6. Quality Control Component

In some embodiments, oligonucleotides undergo a quality control assay before distribution to the user. The specific quality control assay chosen depends on the final use of the oligonucleotides. For example, if the oligonucleotides are to be used in an INVADER SNP detection assay, they are tested in the assay before distribution.

In some embodiments, each SNP set is tested in a quality control assay utilizing the Beckman Coulter SAGIAN CORE System. In some embodiments, the results are read on a real-time instrument (e.g., a ABI 7700 fluorescence reader). The QC assay uses two no target blanks as negative controls and five untyped genomic samples as targets. For consistency, every SNP set is tested with the same genomic samples. In preferred embodiment, the ADS system is responsible for tracking tubes through the QC module. Thus, in some embodiments, if a tube is missing, the ADS program discards, reorders, or searches for the missing tube.

In some preferred embodiments, the user chooses which QC method to run. The operator then chooses how many sets are needed. Then, in some embodiments, the application auto-selects the correct number of SNPs based on priority and prints output (picklist). If a picklist needs to be regenerated, the operator inputs which picklist they are replacing as well as which sets are not valid. The system auto-selects the valid SNPs plus replacement SNPs and print output. Additionally, in some embodiments, picklists are manually generated by SNP number.

The auto-selected SNPs are then removed from being listed as available for auto-selection. In some embodiments, the software prints the following items: SNP/Oligo list (picklist), SNP/Oligo layout (rack setup). The operator then takes the picklist into inventory and removes the completed oligonucleotide sets. In some embodiments, a completed set is unavailable. In this case, the operator regenerates a picklist. Then, in preferred embodiments, the missing SNP set or tube is flagged in the system. Once a picklist is full, the oligonucleotides are moved to the next step.

In some embodiments, the operator then takes the rack setup generated by the picklist and loads the rack. Alternatively, a robotic handling system loads the rack. In preferred embodiments, tubes are scanned as they are placed onto the rack. The scan checks to make sure it is the correct tube and displays the location in the rack where the tube is to be placed.

Completed racks are then placed in a holding area to await the robot prep and robot run. Then, in some embodiments, the operator views what racks are in the queue and determines what genomics and reagent stock will be loaded onto the robot. The robot is then programmed to perform a specific method. Additionally, in some embodiments, the robot or operator records genomics and reagents lot numbers.

In preferred embodiments, a carousel location map is printed that outlines where racks are to be placed. The operator then loads the robot carousel according to the method layout. The rack is scanned (e.g., by the operator or by the ADS program). If the rack is not valid for the current robot method, the operator will be informed. The carousel location for the rack is then displayed. The output plates are then scanned (e.g., by the operator or by the ADS program). If the plate is not valid for the current method the operator is informed. The carousel location for the plate is then displayed.

Then, in some embodiments, the robot is run. The robot then places the plates onto heatblocks for a period of time specified in the method. In some embodiments, the robot then scans the plates on the Cytofluor. Output from the cytofluor is read into the database and attached to the output plate record.

In other embodiments, the output is read on the ABI 7700 real time instrument. In some embodiments, the operator loads the plate on to the 7700. Alternatively, in other embodiments, the robot loads the plate onto the ABI 7700. A scan is then started using the 7700 software. When the scan is completed the output file is saved onto a computer hard drive. The operator then starts the application and scans in the plate bar code. The software instructs the user to browse to the saved output file. The software then reads the file into the database and deletes the file (or tells the operator to delete the file).

The plate reader results (e.g., from a Cytofluor or a ABI 7700) are then analyzed (e.g., by a software program or by the operator). Additionally, in some embodiments, the operator reviews the results of the software analysis of each SNP and takes one of several actions. In some embodiments, the operator approves all automated actions. In other embodiments, the operator reviews and approves individual actions. In some embodiments, the operator marks actions as needing additional review. Alternatively, in other embodiments, the operator passes on reviewing anything. Additionally, in some embodiments, the operator overrides all automated actions.

Depending on the results of the QC analysis, one of several actions is next taken. If the software marks ready for Full Fill, the operator forwards discards diluted Probe/INVADER oligonucleotide mixes and forwards the samples to the packaging module.

If an oligonucleotide set fails quality control, the data is interpreted to determine the cause of the failure. The course of action is determined by such data interpretation. If the software marks an oligonucleotide Reassess Failed Oligonucleotide, no action by user is required, the reassess is handled by automation. In the software marks an oligonucleotide Redilute Failed Oligonucleotide, the operator discards diluted tubes. No other action is required. If the software marks an oligonucleotide Order Target Oligonucleotide, no action by user is required. In this case, a synthetic target oligonucleotide is ordered for further testing. If the software marks an oligonucleotide Fail Oligo(s) Discard Oligo(s), the operator discards the diluted tubes and un-diluted tubes. No other action is required. If the software marks an oligonucleotide Fail SNP, the operator discards the diluted and un-diluted tubes. No other action is required. If the software marks an oligonucleotide Full SNP Redesign, the operator discards the diluted and un-diluted tubes. No other action is required. If the software marks an oligonucleotide Partial SNP Redesign the operator discards diluted tubes and discards some un-diluted tubes. No other action is required.

In some embodiments, the software marks an oligonucleotide Manual Intervention. This step occurs if the operator or software has determined the SNP requires manual attention. This step puts the SNP “on hold” in the tracking system while the operator investigates the source of the failure.

When a set of oligonucleotides (e.g., a INVADER assay set) is completed, the set is transferred to the packaging station.

In some embodiments of the present invention, the produced detection assays are tested against a plurality of samples representing two or more different alleles (samples containing sequences from individuals with different ethnic backgrounds, disease states, etc.) to demonstrate the viability of the assay with different individuals. In preferred embodiments, the produced assays are tested against a sufficient number of alleles (e.g., 100 or more) to identify which members of the population can be tested by the assay and to identify the allele frequency in the population of the genotype for which the assay is designed. In some embodiments, where certain individuals or classes of individuals are not detected by the detection assay, the target sequence of the individuals is characterized to determine whether the intended SNP is not present and/or whether additional mutations are present the prevent the proper detection of the sample. Any such information may be collected and stored in databases. In some embodiments, target selection, in silico analysis, and oligonucleotide design are repeated to generate assays capable of detecting the corresponding sequence of these individuals, as desired. In some embodiments, allele frequency information is stored in a database and made available to users of the detection assays upon request (e.g., made available over a communication network).

C. Packaging Component

In some embodiments, one or more components generated using the system of the present invention are packaged using any suitable means. In some embodiments, the packaging system is automated. In some embodiments, the packaging component is controlled by the centralized control network of the present invention.

D. Centralized Control Network

In some embodiments, the automated DNA production process further comprises a centralized control system. In some embodiments, the centralized control system comprises a computer system.

In some embodiments, the computer system comprises computer memory or a computer memory device and a computer processor. In some embodiments, the computer memory (or computer memory device) and computer processor are part of the same computer. In other embodiments, the computer memory device or computer memory are located on one computer and the computer processor is located on a different computer. In some embodiments, the computer memory is connected to the computer processor through the Internet or World Wide Web. In some embodiments, the computer memory is on a computer readable medium (e.g., floppy disk, hard disk, compact disk, DVD, etc). In other embodiments, the computer memory (or computer memory device) and computer processor are connected via a local network or intranet. In certain embodiments, the computer system comprises a computer memory device, a computer processor, an interactive device (e.g., keyboard, mouse, voice recognition system), and a display system (e.g., monitor, speaker system, etc.).

In preferred embodiments, the systems and methods of the present invention comprise a centralized control system, wherein the centralized control system comprises a computer tracking system. As discussed above, the items to be manufactured (e.g. oligonucleotide probes, targets, etc) are subjected to a number of processing steps (e.g. synthesis, purification, quality control, etc). Also as discussed above, various components of a single order (e.g. one type of SNP detection kit) are manufactured in separate tubes, and may be subjected to a different number of processing steps. Consequently, the present invention provides systems and methods for tracking the location and status of the items to be manufactured such that multiple components of a single order can be separately manufactured and brought back together at the appropriate time. The tracking system and methods of the present invention also allow for increased quality control and production efficiency.

In some embodiments, the computer tracking system comprises a central processing unit (CPU) and a central database. The central database is the central repository of information about manufacturing orders that are received (e.g. SNP sequence to be detected, final dilution requirements, etc), as well as manufacturing orders that have been processed (e.g. processed by software applications that determine optimal nucleic acid sequences, and applications that assign unique identifiers to orders). Manufacturing orders that have been processed may generate, for example, the number and types of oligonucleotides that need to be manufactured (e.g. probe, INVADER oligonucleotide, synthetic target), and the unique identifier associated with the entire order as well as unique identifiers for each component of an order (e.g. probe, INVADER oligonucleotide, etc). In certain embodiments, the components of an order proceed through the manufacturing process in containers that have been labeled with unique identifiers (e.g. bar coded test tubes, color coded test tubes, etc.).

In certain embodiments, the computer tracking system further comprises one or more scanning units capable of reading the unique identifier associated with each labeled container. In some embodiments, the scanning units are portable (e.g. hand held scanner employed by an operator to scan a labeled container). In other embodiments, the scanning units are stationary (e.g. built into each module). In some embodiments, at least one scanning unit is portable and at least one scanning unit is stationary (e.g. hand held human implemented device).

Stationary scanning units may, for example, collect information from the unique identifier on a labeled container (i.e. the labeled container is ‘red’) as it passes through part of one of the production modules. For example, a rack of 100 labeled containers may pass from the purification module to the dilute and fill module on a conveyor belt or other transport means, and the 100 labeled containers may be read by the stationary scanning unit. Likewise, a portable scanning unit may be employed to collect the information from the labeled containers as they pass from one production module to the next, or at different points within a production module. The scanning units may also be employed, for example, to determine the identity of a labeled container that has been tested (e.g. concentration of sample inside container is tested and the identity of the container is determined).

The scanning units are capable of transmitting the information they collect from the labeled containers to a central database. The scanning units may be linked to a central database via wires, or the information may be transmitted to the central database. The central database collects and processes this information such that the location and status of individual orders and components of orders can be tracked (e.g. information about when the order is likely to complete the manufacturing process may be obtained from the system). The central database also collects information from any type of sample analysis performed within each module (e.g. concentration measurements made during dilute and fill module). This sample analysis is correlated with the unique identifiers on each labeled container such that the status of each labeled container is determined. This allows labeled containers that are unsatisfactory to be removed from the production process (e.g. information from the central database is communicated to robotic or human container handlers to remove the unsatisfactory sample). Likewise, containers that are automatically removed from the production process as unsatisfactory may be identified, and this information communicated to a central database (e.g. to update the status of an order, allow a re-order to be generated, etc). Allowing unsatisfactory samples to be removed prevents unnecessary manufacturing steps, and allows the production of a replacement to begin as early as possible.

As mentioned above, the tracking system of the present invention allows the production of single orders that have multiple components that may proceed through different production modules, and/or that may be processed (at least in part) in separate containers. For example, an order may be for the production of an INVADER detection kit. An INVADER detection kit is composed of at least 2 components (the INVADER oligonucleotide, and the downstream probe), and generally includes a second downstream probe (e.g. for a different allele), and one or two synthetic targets so controls may be run (i.e. an INVADER kit may have 5 separate oligonucleotide sequences that need to be generated). The generation of separate sequences, in separate containers, generally necessitates that the tracking system track the location and status of each container, and direct the proper association of completed oligonucleotides into a single container or kit. Providing each container with a unique identifier corresponding to a single type of oligonucleotide (e.g. an INVADER oligonucleotide), and also corresponding to a single order (a SNP detection kit for diagnosing a certain SNP) allows separate, high through-put manufacture of the various components of a kit without confusion as to what components belong with each kit.

Tracking the location and status of the components of a kit (e.g. a kit composed of 5 different oligonucleotides) has many advantages. For example, near the end of the purification module HPLC is employed, and a simple sample analysis may be employed on each sample in each container to determine if a sample is collected in each tube. If no sample is collected after HPLC is performed, the unique identifier on the container, in connection with the central database, identifies the type of sample that should have been produced (e.g. INVADER oligonucleotide) and a re-order is generated. Identification of this particular oligonucleotide allows the manufacturing process for this oligonucleotide to start over from the beginning (e.g. this order gets priority status over other orders to begin the manufacturing process again). Importantly, the other components of the order may continue the manufacturing process without being discarded as part of a defective order (e.g. the manufacturing process may continue for these oligonucleotides up to the point where the defective oligonucleotide is required). Likewise, additional manufacturing resources are not wasted on the defective component (i.e. additional reagents and time are not spent on this portion of the order in further manufacturing steps).

The unique identifier on each of the containers allows the various components of a given order to be grouped together at a step when this is required (likewise, there is no need to group the components of an order in the manufacturing process until it is required). For example, prior to the dilute and fill module, the various components of a single order may be grouped together such that the contents of the proper containers are combined in the proper fashion in the dilute and fill module. This identification and grouping also allows re-orders to ‘find’ the other components of a particular order. This type of grouping, for example, allows the automated mixing, in the dilute and fill stage, of the first and second downstream probes with the INVADER oligonucleotide, all from the same order. This helps prevent human errors in reading containers and accidentally providing probes intended for one SNP being labeled as specific for a different SNP (i.e. this helps prevent components of different kits from being accidentally mixed together). The identification of individual containers not only allows for the proper grouping of the various components of a single order, but also allows for an order to be customized for a particular customer (e.g. a certain concentration or buffer employed in the second dilute and fill procedure). Finally, containers with finished products in them (e.g. containers with probes, and containers with synthetic targets) need to be associated with each other so they are properly assayed in the quality control module, and packaged together as a single kit (otherwise, quality control and/or a final end-user may find false negative and false positives when attempting to test/use the kit). The ability to track the individual containers allows the components of a kit to be associated together by directing a robot or human operator what tubes belong together. Consequently, final kits are produced with the proper components. Therefore, the tracking systems and methods of the present invention allow high through-put production of kits with many components, while assuring quality production.

E. Example

This Example describes the production of an INVADER assay kit for SNP detection using the automated DNA production system of the present invention.

1. Oligonucleotide Design

The sequence of the SNP to be detected is first submitted through the automated web-based user interface or through e-mail. The sequences are then transferred to the INVADER CREATOR software. The software designs the upstream INVADER oligonucleotide and downstream probe oligonucleotide. The sequences are returned to the user for inspection. At this point, the sequences are assigned a bar code and entered into the automated tracking system. The bar codes of the probe and INVADER oligonucleotide are linked so that their synthesis, analysis, and packaging can be coordinated.

2. Oligonucleotide Synthesis

Once the probe and INVADER oligonucleotide sequences have been designed, the sequences are transferred to the synthesis component. The bar codes are read and the sequences are logged into the synthesis module. Each module consists of 14 MOSS EXPEDITE 16-channel DNA synthesizers (PE Biosystems, Foster City, Calif.), that prepare the primary probes, and two ABI 3948 48-Channel DNA synthesizers (PE Biosystems, Foster City, Calif.), that prepare the INVADER oligonucleotides. Synthesizing a set of two primary and INVADER probes is complete 3-4 hours. The instruments run 24 h/day. Following synthesis, the automating tracking system reads the bar codes and logs the oligonucleotides as having completed the synthesis module.

The synthesis room is equipped with centralized reagent delivery. Acetonitrile is supplied to the synthesizers through stainless steel tubing. De-blocking solution (3% TCA in methylene chloride) is supplied through Teflon tubing. Tubing is designed to attach to the synthesizers without any modification of the synthesizers. The synthesis room is also equipped with an automated waste removal system. Waste containers are equipped with ventilation and contain sensors that trigger removal of waste through centralized tubing when the cache pots are full. Waste is piped to a centralized storage facility equipped with a blow out wall. The pressure in the synthesis instruments is controlled with argon supplied through a centralized system. The argon delivery system includes local tanks supplied from a centralized storage tank.

During synthesis, the efficiency of each step of the reaction is monitored. If an oligonucleotide fails the synthesis process, it is re-synthesized. The bar coding system scans the container of the oligonucleotide and marks it as being sent back for re-synthesis.

Following synthesis, the oligonucleotides are transported to the cleavage and deprotection station. At this stage, completed oligonucleotides are subjected to a final deprotection step and are cleaved from the solid support used for synthesis. The cleavage and deprotection may be performed manually or through automated robotics. The oligonucleotides are cleaved from the solid support used for synthesis by incubation with concentrated NaOH and collected. The cleavage step takes 12 hours. Following cleavage, the bar code scanner scans the oligonucleotide tubes and logs them as having completed the cleavage and deprotection step.

3. Purification

Following synthesis and cleavage, probe oligonucleotides are further purified using HPLC. INVADER oligonucleotides are not purified, but instead proceed directly to desalting (see below).

HPLC is performed on instruments integrated into banks (modules) of 8. Each HPLC module consists of a Leap Technologies 8-port injector connected to 8 automated Beckman-Coulter HPLC instruments. The automatic Leap injector can handle four 96-well plates of cleaved and deprotected primary probes at a time. The Leap injector automatically loads a sample onto each of the 8 HPLCs.

Buffers for HPLC purification are produced by the automated buffer preparation system. The buffer prep system is in a general access area. Prepared buffer is then piped through the wall in to clean room (HEPA environment). The system includes large vat carboys that receive premeasured reagents and water for centralized buffer preparation. The buffers are piped from central prep to HPLCs. The conductivity of the solution in the circulation loop is monitored as a means of verifying both correct content and adequate mixing. The circulation lines are fitted with venturis for static mixing of the solutions; additional mixing occurs as solutions are circulated through the piping loop. The circulation lines are fitted with 0.05 μm filters for sterilization and removal of any residual particulates.

Each purified probe is collected into a 50-ml conical tube in a carrying case in the fraction collector. Collection is based on a set method, which is triggered by an absorbance rate change within a predetermined time window. The HPLC is run at a flow rate of 5-7.5 ml/min (the maximum rate of the pumps is 10 ml/min.) and each column is automatically washed before the injector loads the next sample. The gradient used is described in Tables 3 and 4 and takes 34 minutes to complete (including wash steps to prepare the column for the next sample). When the fraction collector is full of eluted probes, the tubes are transferred manually to customized racks for concentration in a Genevac centrifugal evaporator. The Genevac racks, containing dry oligonucleotide, are then transferred to the TECAN Nap10 column handler for desalting.

4. Desalting

Following HPLC purification (probe oligonucleotides) or cleavage (INVADER oligonucleotides), oligonucleotides move to the desalting station. The dried oligonucleotides are resuspended in a small volume of water. Desalting steps are performed by a TECAN robot system. The racks used in Genevac centrifugation are also used in the desalting step, eliminating the need for transfer of tubes at this step. The racks are also designed to hold the different sizes of desalting columns, such as the NAP-5 and NAP-10 columns. The TECAN robot loads each oligonucleotide onto an individual NAP-5 or NAP-10 column, supplies the buffer, and collects the eluate.

5. Dilution

Following desalting, the oligonucleotides are transferred to the dilute and fill module for concentration normalization and dispenation. Each module consists of three automated probe dilution and normalization stations. Each station consists of a network-linked computer and a Biomek 2000 interfaced with a SPECTRAMAX spectrophotometer Model 190 or PLUS 384 (Molecular Devices Corp., Sunnyvale Calif.) in a HEPA-filtered environment.

The probe and INVADER oligonucleotides are transferred onto the Biomek 2000 deck and the sequence files are downloaded into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses the measured absorbance and the sequence information to calculate the concentration of each oligonucleotide. The software then prepares a dilution table for each oligonucleotide. The probe and INVADER oligonucleotide are each diluted by the Biomek to a concentration appropriate for their intended use. The instrument then combines and dispenses the probe and INVADER oligonucleotides into 1.5 ml microtubes for each SNP set. The completed set of oligonucleotides contains enough material for 5,000 SNP assays.

If an oligonucleotide fails the dilution step, it is first re-diluted. If it again fails dilution, the oligonucleotide is re-purified or returned for re-synthesis. The progress of the oligonucleotide through the dilution module is tracked by the bar coding system. Oligonucleotides that pass the dilution module are scanned as having completed dilution and are moved to the next module.

6. Quality Control

Before shipping, the SNP set is subjected to a quality control assay in a SAGIAN CORE System (Beckman Coulter), which is read on a ABI 7700 real time fluorescence reader (PE Biosystems). The QC assay uses two no target blanks as negative controls and five untyped genomic samples as targets.

The quality control assay is performed in segments. In each segment, the operator or automated system performs the following steps: log on; select location; step specific activity; and log off. The ADS system is responsible for tracking tubes. If a tube is missing, existing ADS program routines will be used to discard/reorder/search for the tube.

In the first step, a picklist is generated. The list includes the identity of the SNPs that are being tested and the QC method chosen. The tubes containing the oligonucleotide are selected by the automated software and a copy of the picklist is printed. The tubes are removed from inventory by the operator and scanned with the bar code reader and being removed from inventory.

The operator or the automated system then takes the rack setup generated by the picklist and loads the rack. Tubes are scanned as they are placed onto the rack. The scan checks to make sure it is the correct tube and displays the location in the rack where the tube is to be placed. Completed racks are placed in a holding area to await the robot prep and robot run.

The operator or the automated system then chooses the genomics and reagent stock to be loaded onto the robot. The robot is programmed with the specific method for the SNP set generated. Lot numbers of the genomics and reagents are recorded. Racks are placed in the proper carousel location. After all the carousel locations have been loaded the robot is run.

Places are then incubated on the robot. The plates are placed onto heatblocks for a period of time specified in the method. The operator then takes the plate and loads it into the ABI 7700. A scan is started using the 7700 software. When the scan is completed the operator transfers the output file onto a Macintosh computer hard drive. The then starts the analysis application and scans in the plate bar code. The software instructs the operator to browse to the saved output file. The software then reads the file into the database and deletes the file.

The results of the QC assay are then analyzed. The operator scans plate in at workstation PC and reviews automated analysis. The automated actions are performed using a spreadsheet system. The automated spreadsheet program returns one of the following results:

1) Mark SNP Oligonucleotide ready for full fill (Operator discards diluted Probe/INVADER mixes. Requires no other action).

2) ReAssess Failed Oligonucleotide (Requires no action by operator, handled by automation).

3) Redilute Failed Oligonucleotide (Operator discards diluted tubes. Requires no other action).

4) Order Target Oligonucleotide (Requires no action by operator, handled by automation).

5) Fail Oligo(s) Discard Oligo(s) (Operator discards diluted tubes. Operator discards un-diluted tubes. Requires no other action).

6) Fail SNP (Operator discards diluted tubes. Operator discards un-diluted tubes. Requires no other action).

7) Full SNP Redesign (Operator discards diluted tubes. Operator discards undiluted tubes. Requires no other action).

8) Partial SNP Redesign (Operator discards diluted tubes. Operator discards some un-diluted tubes. Requires no other action).

9) Manual Intervention (This step occurs if the operator or software has determined the SNP requires manual attention. This step puts the SNP “on hold” in the tracking system).

The operator then views each SNP analysis and either approves all automated actions, approves individual actions, marks actions as needing additional review, passes on reviewing anything, or over rides automated actions.

Once the SNP set has passed the QC analysis, the oligonucleotides are transferred to the packaging station.

In some embodiments, the produced detection assay is screened against a plurality of known sequences designed to represent one or more population groups, e.g., to determine the ability of the detection assay to detect the intended target among the diverse alleles found in the general population. In preferred embodiments, the frequency of occurrence of the SNP allele in each of the one or more population groups is determined using the produced detection assay. Data collected may be used to satisfy regulatory requirements, if the detection assay is to be used as a clinical product.

IV. Detection Assay Use and Data Generation and Collection

While the above sections describe the generation of a detection assay and the validation of the assay against a number of samples (e.g., several hundred samples), to fully investigate the viability of the detection assay against a broader population it is sometimes desired to conduct widespread testing with the detection assay. Where many different detection assays (e.g., hundreds to thousands of detection assays designed to identify unique markers) are to be investigated to facilitate moving products from research markets to clinical markerts, large numbers of detection assays are tested against large numbers of samples. In some embodiments, a detection assay producer distributes detection assays to research collaborators, whereby the research collaborators each conduct large numbers of tests (e.g., because of the inability of any one party to carry out a sufficient number of tests). The data generated by these tests is used to validate the detection assay (e.g., for use in obtaining regulatory approval). Test results may show that the detection assay is suitable or not suitable for use in certain population sub-sets. The test results may also show that detection assays, for whatever reason (e.g., for determined or undetermined scientific reasons), are not suitable for one or more testing markets (e.g., do not provide the requisite data to achieve regulatory approval). Where tests are determined not suitable for a desired market, new tests may be generated using the methods described above to identify a candidate test that meets the desired criteria.

In some embodiments, a detection assay directed to a single target is used. However, in certain preferred embodiments, panels containing a plurality of different detection assays are employed (e.g., produced and used in testing). For example, panels containing two or more markers associated with a particular medical condition are employed. In some preferred embodiments, the panels contain thousands of unique markers, corresponding to every identified medically relevant marker.

The present invention provides systems and methods to provide researchers using the detection assays with information to assist in data collection as well as system and methods to collect and analyze data. In particularly preferred embodiments, collected data is automatically directed to a processor for analysis, storage, and compilation (e.g., compilation to support an application requesting regulatory approval of clinical products).

In some such embodiments, the present invention provides users with a means to find known information (including but not limited to information gleaned from public sources, publications, patents, and information previously determined by any user of the database) about any SNP, other mutations, or other sequence characteristic that has been entered a database. In some embodiments, the present invention provides a facile means of linking known and collected information about a particular SNP, other mutations, or other sequence characteristic to a particular test (e.g., assay test) of a sample. The utility of such applications is illustrated below for embodiments where SNP information is to be analyzed.

Association Databases

When a SNP has been linked to any other item of information (e.g., disease state, chromosome location, gene, ethnic group, allele frequency, another SNP), it can be considered to have an association. Association databases may be configured with reference to any association or combination of associations. In a preferred embodiment, an association database is configured to contain information about SNPs that have been determined to have medical relevance (i.e., to be relevant to some aspect of health, including but not limited to the presence of disease, disease susceptibility and prognosis, and individual response to particular therapy).

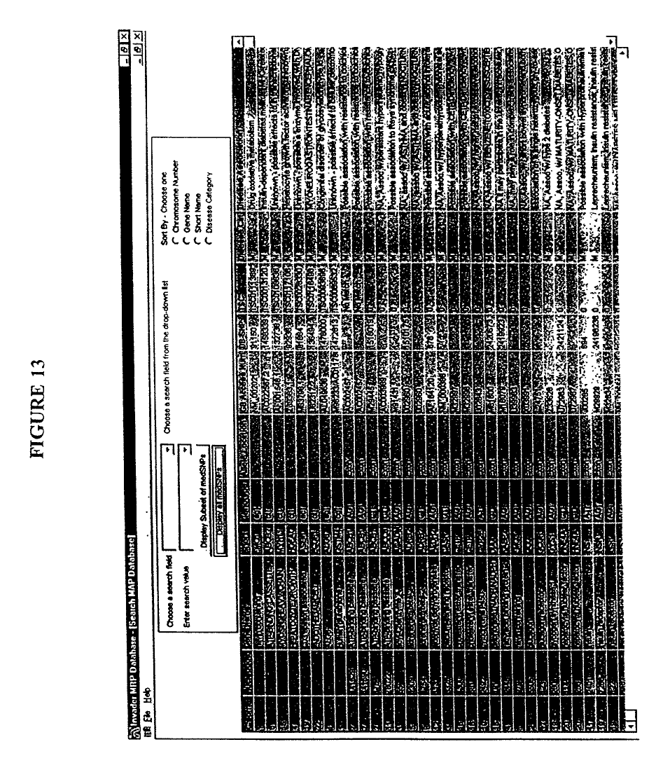

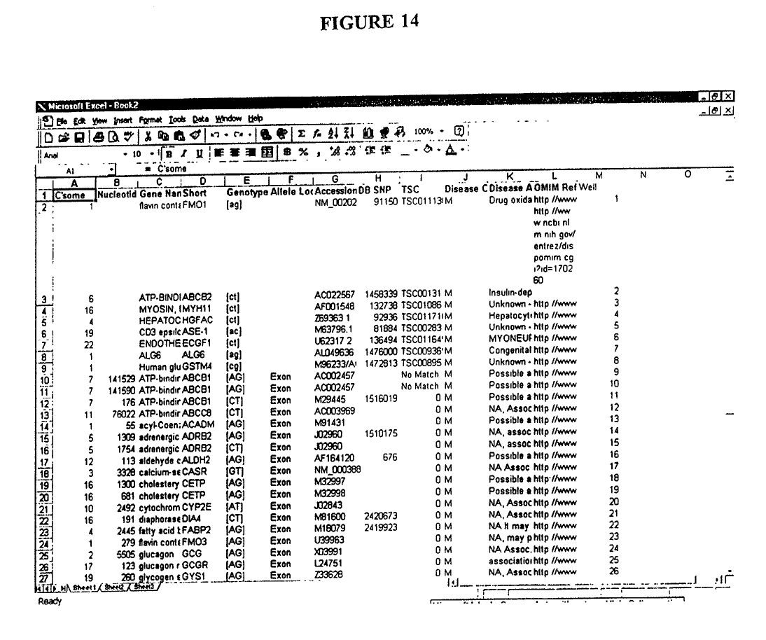

In one embodiment, information about a SNP can be provided in a database table (e.g., a Microsoft Access database) having alphanumeric fields to provide details such as the gene identification, medical relevancy of the polymorphism, and literature or other references for the information provided (FIG. 13). Any number of fields are contemplated. In some embodiments, information may be as simple as a single gene name or an accession number in a database (e.g., GenBank). In other embodiments, the fields may provide more information, including but not limited to chromosome number, nucleotide, gene name, gene name abbreviation, genotype designation, allele location, GenBank accession number, NCBI URL link, dbSNP number, TSC number, targeted DNA sequence, disease category, disease association(s), SNP association(s) (i.e., other SNPs or mutations found to be associated the SNP being reviewed), patent status (e.g., whether a patent relating to that SNP has been identified), patent number(s), and the NCBI OMIM database URL link. Additional links or items of information may be provided, such as links to online reference libraries and patent or other intellectual property databases. Disease categories may include, for example, metabolism, endocrinology, pulminology, nephrology, gastroenterology, neurology, genetic disease, musculoskeletal, and immunology. Additional categories may be designated to specifically identify diseases that overlap into two or more particular categories. Yet another kind of category may be provided (e.g., a “miscellaneous” category) for SNPs that have unknown or indeterminate association, that have a known association that does not fall within another category, or that, for any other reason, are not appropriately assigned to another category. In some embodiments the database has one field. In preferred embodiments the database has at least 10 fields, and in a particularly preferred embodiment, the database has at least 20 fields. In some embodiments, the database table is displayed on a screen (FIG. 13). In preferred embodiments, the screen is printable. In some embodiments, the fields are exportable to a spreadsheet file or worksheet (e.g., in Microsoft Excel; FIG. 14).

In one embodiment, the database may be searchable. In a preferred embodiment, the database is searchable, and is also configured to allow the user to present the resulting search data sets in an easily understandable, meaningful manner. In some embodiments, the database comprises an “allele caller” function, a function that provides allele calls (i.e., identification of the alleles detected in a given assay) based on the data input (e.g., such as from a fluorescent reader or mass spectrometer).

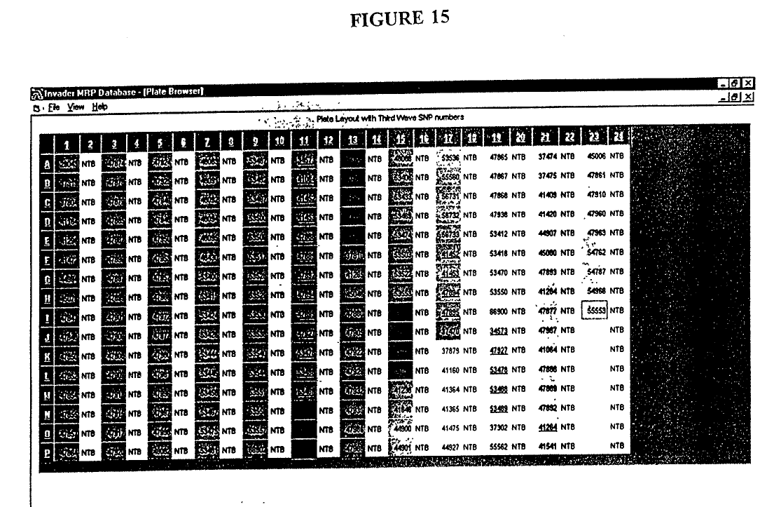

In some embodiments, the present invention provides a means for easily linking known information about a particular SNP to a particular test result on a sample through a “plate viewer” format corresponding to the layout of samples in a reaction vessel or plate (FIG. 15). In preferred embodiments, the present information provides a means to use particular SNP test results on a sample to amend or update information about that SNP in an association database.

The following discussion provides one example of how a user interface for an association database may be configured. The user opens a work screen by clicking on an icon on a desktop display of a computer (e.g., a Windows desktop). The work screen features a menu (e.g., a drop down menu or “options” buttons) that allows the user to choose from available options. For example, in one embodiment, a user may be presented with the options of: 1) searching an association database; or 2) opening a plate viewer (as described above). In other embodiments, the user may have further or different options, such as 3) running an allele caller function. An option for exiting the program may be provided on the menu, as well. Examples of possible embodiments of user interfaces for each of these options are described, below.

1. Searching an Association Database:

In one embodiment, selecting this option opens a form having boxes that allow the user to make alphanumeric entries, and/or combination boxes (e.g., boxes that allow the user to either select from a list or make an alphanumeric entry) for each field represented in that particular association database. The user can enter search criteria in any field or set of fields. Upon clicking a “search” button, the program constructs a query, searching for record sets that include the specified strings in the corresponding fields.

Matching records from the search are assembled into sets. In some embodiments, the matching sets are displayed on a screen. In other embodiments, the matching sets are exported (e.g., sent to a printer or a file, or to a further process step) without display. In a preferred embodiment, the matching sets are displayed in a printable window.

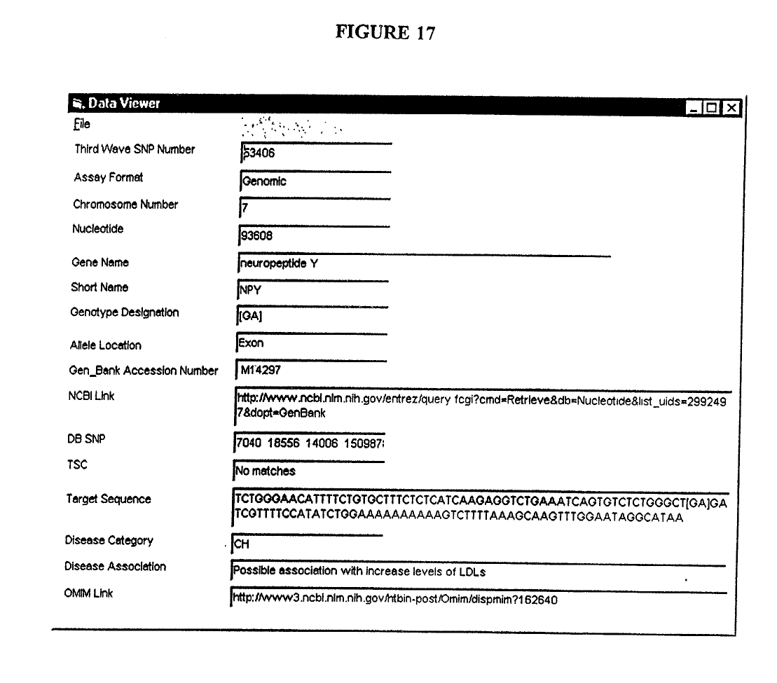

In some embodiments, the user may select an entry from the matching set and view the information in the fields. In some embodiments, selection of an entry creates a display of the fields for that entry (FIG. 17). In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields contain one or more local or Internet links (e.g., hypertext links or URLs). In preferred embodiments, SNPs listed in a SNP association field provide links to the record(s) of the associated SNPs. In particularly preferred embodiments, the user can click on links to bring up the corresponding content.

2) Using a Plate Viewer

As noted above, the present invention provides a means for easily linking known information about a particular SNP to a particular test result on a sample through a “plate viewer” format, i.e., in a fashion that corresponds to (e.g., visually represents) the layout of samples in a reaction vessel (FIG. 15). For example, if test assays for SNPs are performed in 96-well microtiter plates, which are arranged in grids of 8 wells×12 wells, the links to the information regarding the SNPs would be displayed in a grid of 8×12 cells, such that each cell corresponds to the particular well in the plate (i.e., the test SNP in the 3^{rd} well of the 4^{th} row will have a link to its information presented on screen in the 3^{rd} cell of the 4^{th} row). Similar displays corresponding to other layouts of reaction vessels are contemplated (e.g., staggered grids, or circular or linear layouts). Any layout that can be replicated as a computer display is contemplated, including any non-gridded, or random distribution of reaction vessels in any arrangement that may be captured for representation on a computer display. Locations may be entered manually, or they may be automatically sensed and entered by methods such as digital imaging, coordinate sensing (e.g., such as that used for touch-screen computer displays), and the like.

Using a 384-well plate, a user selecting a “Plate Viewer” option should be presented with a table in the 384-well plate layout. In one embodiment, the SNPs entered into each cell of the table are assigned by the user (e.g., by entering identifying information from a particular field, such as a dbSNP number, into a selected cell on the plate viewer table). In preferred embodiments, SNPs are pre-assigned to particular cells. In particularly preferred embodiments, the SNPs are pre-assigned to cells in the table such that they correspond with an assay plate configured to test those SNPs in the corresponding wells. In other particularly preferred embodiments, the user selects from a menu of Plate Viewers, each having a different set of SNPs in pre-assigned cells corresponding with an assay plate configured to test those SNPs in the corresponding wells.

In one embodiment, the user selects which field of the SNP record assigned to that cell will be displayed in the cell. In some embodiments, different fields from each SNP record may be displayed in each of the different cells. In other embodiments, the cells are coordinated so that the same field from each SNP record is displayed in each assigned cell. In a preferred embodiment, the user can globally change the fields displayed in all cells (e.g., through the use of a menu), such that all of the cells can be changed at one time to display the same field from each different SNP record.

In some embodiments, there is a code to visually distinguish test SNPs from control reactions (e.g., ‘no target’ controls or other controls). In preferred embodiments, the code is a color code.

In some embodiments, the user may select an entry from a cell and view (e.g., in a “data viewer”) the information in all of the fields for that SNP record (FIG. 17). In some embodiments, selection of an entry creates a display of the fields for that entry. In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields contain one or more local or Internet links (e.g., hypertext links or URLs). In preferred embodiments, the user can click on links to bring up the corresponding content.

In some embodiments, an association database is provided on removable storage media (e.g., compact disc). In further embodiments, the storage media having the database includes an index of any PlateViewers having pre-assigned SNP records contained thereon. In preferred embodiments, the storage media having the database provides an indication of the currency of the information in the recorded database (e.g., a date or date range, version number, etc.). In preferred embodiments, the storage media having the database provides contact information for technical support (e.g., phone numbers facsimile numbers, email addresses, street addresses, names of technical support personnel, etc.).

3) Running an Allele Caller Function.

In some embodiments, the association database comprises an “allele caller” function, a function that provides identification of the alleles detected in a given assay, based on input assay data (e.g., from an instrument such as a fluorescent reader, nucleic acid chip reader, or mass spectrometer).

The data to be processed by an allele caller may be provided in many different forms. In some embodiments, the data is raw signal, such as number corresponding to a measurement of fluorescence signal from a spot on a chip or a reaction vessel, or a number corresponding to measurement of a peak (e.g., peak height or area, as from, for example, a mass spectrometer, HPLC or capillary separation device). In some embodiments the data is imported directly from a measuring device. In other embodiments, the data is imported from a file. Raw data may be generated by any number of SNP detection methods, including but not limited to those listed below.

1. Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. In preferred emodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

4. Hybridization Assays

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

The INVADER assay detects specific mutations and SNPs in unamplified genomic DNA. In an embodiment of the INVADER assay used for detecting SNPs in genomic DNA, two oligonucleotides (a primary probe specific either for a SNP/mutation or wild type sequence, and an INVADER oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin).

5. Mass Spectroscopy Assay

In some embodiments, a MassARRAY system (Sequenom, San Diego, Calif.) is used to detect variant sequences (See e.g., U.S. Pat. Nos. 6,043,031; 5,777,324; and 5,605,798; each of which is herein incorporated by reference). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

In some embodiments, data generated by different detection methods are processed to facilitate comparison, e.g., using an process like the Extraction-Transformation-Load paradigm from Data Warehousing, wherein data is “published” into a single repository, normalizing disparate data, and optimizing it for browsing and easy access to normalized, integrated data (e.g., DataMart and MetaSymphony software, NetGenics, Inc., Cleveland Ohio; U.S. Pat. No. 6,125,383, incorporated herein by reference in its entirety). SNP data generated by one SNP analysis method may be compared to SNP results data generated by another SNP analysis method (e.g., INVADER assay results are compared to gene chip data).

In some embodiments of the present invention, data is processed using an algorithm selected to determine an allele from the input assay data. The algorithm selected for processing data may be determined by the nature of the input assay data. The following provides an example of the application of an allele caller to an assay run in a microtiter plate (e.g., a 384-well plate).

The user enters information to identify the plate to be analyzed. In one embodiment, the plate may be identified by entry of a code number (e.g., a barcode number, part number, lot number). In another embodiment, the program provides a menu from which the user selects the number corresponding to the plate.

In some embodiments, the program provides a validation of the plate. For example, in some embodiments, the program verifies that the plate is of a suitable format for available analysis (e.g., that it corresponds to an assay for which an allele caller function can be provided). In other embodiments, the program verifies that the plate has been passed through some other process step. In some embodiments wherein the association database is provided on removable media (e.g., as described above), the program verifies that the version of the CD in use is suitable (e.g., has an appropriate version of an allele caller function, or has an appropriate association database) for use with the plate to be analyzed.

When a plate has been identified and determined to be valid for analysis, a record is displayed. In preferred embodiments, the record is a table having cells that correspond to assay wells on a microtiter plate (e.g., a “plate viewer”, described above). In some embodiments, the user has the option (e.g., through a menu selection) of creating a new analysis record or of calling up a record of a prior analysis. In preferred embodiments, the record links to identifying data from other analyses performed on the same collection of samples (e.g., name, date generated, etc.). In particularly preferred embodiments, SNP test wells on a plate are linked through a “plate viewer” function to SNP records in a database. In further particularly preferred embodiments, the database is an association database.

Prior to analysis, the assay data from the plate is imported, or “loaded” into the analysis program. It is contemplated that the data to be processed by an allele caller may be provided in many different forms. In some embodiments, the assay data is raw (i.e., unanalyzed) signal, such as a number corresponding to a measurement of fluorescence signal from a spot on a chip or a reaction vessel, or a number corresponding to measurement of a peak (e.g., peak height or area, as from, for example, a mass spectrometer, HPLC or capillary separation device). In some embodiments the data is imported directly from a measuring device. In other embodiments, the data is imported from a file. Raw assay data may be generated by any number of SNP detection methods, including but not limited to those listed above.

In some embodiments, the loaded assay data is displayed on a screen. In preferred embodiments, data is displayed in a plate viewer format. In some preferred embodiments, the layout is displayed in a new window. In particularly preferred embodiments, the window is printable.

Loaded assay data is then analyzed or processed using one or more algorithms selected to determine an allele from the input assay data. The algorithm selected for processing data is generally determined by the nature of the input assay data. In some embodiments, analysis involves determining the presence or absence of a signal (e.g., detectable fluorescence, or a detectable peak). In other embodiments, analysis involves determining the presence of a signal meeting a threshold value. In still other embodiments, analysis involves a comparison of more than one signal (e.g., examining differences in signal level, calculating ratios, etc.). In preferred embodiments, a SNP result (i.e., a determination of genotype at that locus, such as homozygous Allele 1 or Allele 2, heterozygous, Indeterminate) is determined when the processed data yields or corresponds to a value that has been predetermined to be indicative of a particular SNP result.

In some embodiments, the SNP results data from one plate are compared with the SNP results data from another plate. In other embodiments, SNP results data generated by one SNP analysis source method are compared to SNP results data generated by another SNP analysis method (e.g., INVADER assay results are compared to gene chip data).

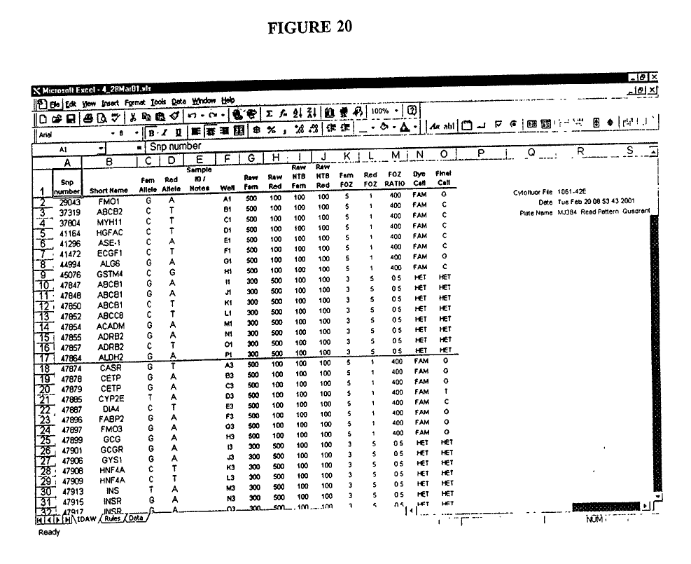

In some embodiments, analysis results are displayed. In other embodiments, the analysis results are exported (e.g., sent to a printer or a file, or to a further process step) without display. In preferred embodiments, SNP results are displayed on a screen. In particularly preferred embodiments, results are displayed in a plate viewer (FIG. 20). In some preferred embodiments, the plate viewer is displayed in a new window. In particularly preferred embodiments, the window is printable.

In some embodiments, the user may select a particular SNP result from the display of results and view the information in fields. In some embodiments, selection of an entry creates a display of the fields for that entry. In some embodiments, all the fields of the SNP record in an association database are shown. In other embodiments, a subset of the fields is shown. In preferred embodiments, fields in SNP results records include but are not limited to results of the analysis (e.g., homozygous Allele 1 or Allele 2, heterozygous, Indeterminate), the entered or imported raw input assay data (e.g., measured fluorescence, measured peaks, etc.), or the analyzed input assay data by which the allele determination was made (e.g., calculated differences in signal level, calculated ratios). In preferred embodiments, a field for user comments is included. In particularly preferred embodiments, the user comment field is editable after a SNP result has been obtained. In further particularly preferred embodiments, changes in a SNP result record may be saved by the user to that record or to a version of that record after a comment field is edited.

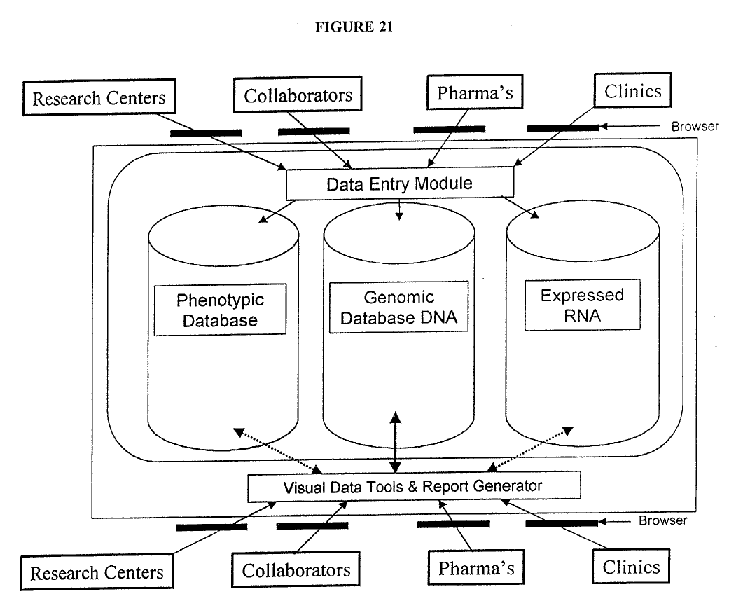

In some embodiments, the user selects which field of the SNP result record assigned to that cell will be displayed in the cell (FIGS. 20 and 21). In some embodiments, different fields from each SNP result record may be displayed in each of the different cells. In other embodiments, the cells are coordinated so that the same field from each SNP result record is displayed in each assigned cell. In a preferred embodiment, the user can globally change the fields displayed in all wells (e.g., through the use of a menu), such that all of the cells can be changed at one time to display the same field from each different SNP result record.

In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields will contain one ore more local or Internet links (e.g., hypertext links or URLs). In preferred embodiments, the user can click on links to bring up the corresponding content.

In some embodiments, there is a code to visually distinguish test SNPs results and control reaction results (e.g., ‘no target’ controls or other controls). In preferred embodiments, the code is a color code.

In some embodiments, the fields are exportable to a spreadsheet file or worksheet (e.g., in Microsoft Excel, FIG. 20). In some embodiments, SNP result data are exported to a worksheet by field content (e.g., one worksheet with all allele calls, one worksheet with all calculated ratios of signals, one worksheet with all raw input fluorescence measurements). In other embodiments, SNP results data are exported, all data is exported to a single worksheet, with data grouped according to the well with which it corresponds. In preferred embodiments, the user has the option (e.g., through a menu or window) of selecting a variety ways in which the SNP results data are sorted and/or grouped for export to a spreadsheet.

In preferred embodiments, following verification, assays for the detection of a given SNP are tested on a plurality of additional individuals. Data from additional assays is combined with information obtained from database searches. In preferred embodiments, the result is a revised reliability score for the SNP. In particularly preferred embodiments, data from additional analysis (e.g., results generated by an investigator using the methods and systems of the present invention) is used to update or amend an association database containing information about the given SNP.

A. Database Software

In some embodiments, GENOMICA (Boulder, Colo.) software is utilized to generate and host the SNP database of the present invention. In some embodiments, GENOMICA DISCOVERY MANAGER software is utilized. Genomica software utililizes Oracle databases to provide a web interface, security features, and reporting information (e.g., including but not limited to, the information described in Section C below). Depending on the particular application, one or more of the features of DISCOVERY MANAGER are utilized.

B. Revisions of Database Information

In preferred embodiments, the information (e.g., reliability scores) in the SNP database of the present invention is revised on a regular basis. In some embodiments, the revisions are automated. For example, users (e.g., customers) provide data from genotyping studies (e.g., through an automated web interface). In some embodiments, individual users are given a reliability rating based on the quality of their genotyping information. In preferred embodiments, the contribution to the reliability score of an individual's data is weighted based on the reliability rating of the user. In addition, individual databases are given reliability ratings based on the verification of their data.

V. Integrated Information, Design, and Production

Data gathered from the use of detection assays on one or more samples (e.g., as described in Section IV, above) may be used to generate and expand powerful genomics databases and to supplement and improve target selections, detection assay design, detection assay productions, and detection assay use, and further analysis of detection assay results. The data may also be used to obtain regulatory approval for clinical products for detection assays that are demonstrated to meet the necessary requirements for clinical regulatory approval (described below). While, for clarity, each of the components of the systems and methods of the present invention has been described herein in isolation, each component relates to each other component, and the synergy between the components provides enhanced systems and methods for acquiring and analyzing biological information. This synergy, as it relates to some embodiments of the present invention, is represented in FIG. 21. The center of the figure shows genomic databases representing phenotypic databases (e.g., disease databases), genomic databases (e.g., genome sequence databases, polymorphism databases, allele frequency databases, etc.), and expressed RNA databases. Data in the databases is derived from any number of sources. For example, the databases may contain data from compiled public or private databases. Data may also be actively incorporated using systems and methods of the present invention. As shown in FIG. 21, data is received from investigators (e.g., using a communication network) providing target sequence requests for in silico analysis, detection assay design, and/or detection assay production (See e.g., Sections I, II, and III, above).

In some embodiments, new data is generated during the processes of the present invention (e.g, produced assays may be tested on a plurality of samples to determine allele frequencies, as described in Section III). New data is also received from detection assay data gathered from investigators (See e.g., Section IV, above). In some embodiments of the present invention, information is tracked and correlated from the initial target sequence requests to the final detection assay result data analysis.

Newly collected data may be incorporated into a number of aspects of the present invention. It can be used to refine in silico analysis, e.g., to provide improved output information; it may be added to an association database, e.g., to note newly observed associations within existing fields, and/or to define new fields indicating new types of associations, such as allele frequency within populations tested.

The following example is provided to illustrate certain preferred embodiments of the present invention. In this example, the systems for performing in silico analysis, detection assay design and production, and information management and analysis are provided by a service provider. Target sequences to be analyzed are provided by a first user (e.g., a researcher, pharmaceutical company, government agency, etc.) and detection assays generated to detect the target sequence are used by the first user and/or other users.

The first user selects a target sequence of interest. For example, an investigator may have identified a SNP in a human genomic sequence that is correlated to disease state (e.g., a SNP correlated to cardiovascular disease, diabetes, development of cancer, rare inherited disorders, asthma, neurological diseases, obesity, sexual dysfunction, hypertension, and the like). In some cases, the investigator will have identified the mutation and/or correlation in a very small population sample (e.g., in a single individual). The investigator may wish to determine the allele frequency of the SNP in the general population and may wish to generate an accurate diagnostic test to determine if an individual possesses the SNP, and is therefore at a higher risk than the general population of contracting or exhibiting the correlated disease or condition. In other embodiments, an investigator may have a SNP that is only suspected to correlate to a disease state, and may wish to generate an accurate diagnostic test to screen large numbers of individuals who have been assessed for the presence or absence of the disease state in order to determine the whether the suspected correlation in fact exists. In other cases, the investigator may wish to determine the frequency of an allele within one or more populations for purposes including assessing risk for correlated disease states in the one or more populations. To address these needs, the investigator employs the systems and methods of the present invention.

The investigator uses a computer system to access a computer system of the service provider. In some embodiments, the investigator simply uses a personal computer system to access a publicly available Web site of the service provider. As discussed in Section I, above, the user transmits the identified target sequence containing the SNP to the computer system of the service provider. The target sequence is then processed through the in silico analysis systems and methods (Section I) and the detection assay design systems and methods (Section II) of the present invention. A report is sent to the investigator indicating any problems identified in the in silico analysis or design process and, in some embodiments, alternate target sequence suggestions are provided. The report may also indicate several options for the design of a detection assay from which the investigator may select. In some embodiments, at the time the original target sequence is submitted by the investigator, the investigator selects options for determining whether a report is provided (e.g., as opposed to simply proceeding with production without generating a report), the conditions under which a report is provided, and the information content of the report.

Once a target sequence is selected and design parameters for the detection assay components are selected (e.g., type of target [RNA or DNA] sequences of probes and primers, reaction temperatures, buffer conditions, etc.), information is passed to the production component of the systems and methods of the present invention (Section III). Production of the detection assay is carried out and quality control steps are used to ensure that the detection assay functions as intended (i.e., is capable of detecting the SNP in a sample). In some embodiments, the produced detection assay is screened against a plurality of known sequences designed to represent one or more population groups, e.g., to determine the ability of the detection assay to detect the intended target amongst the diverse alleles found in the general population. Produced assays are then shipped to detection assay users (e.g., the investigator who entered the target sequence and other investigators).

At each of the stages described above, information is tracked and stored. For example, the original target sequence request from the investigator is assigned a tracking number and information about the investigator (e.g., previous request information), information obtained from in silico analysis, information obtained from design analysis, and information obtained from production analysis (e.g., allele frequency information) is collected, correlated to the tracking number, and incorporated into the databases of the present invention. For example, allele frequency information is stored in a SNP allele frequency database, information obtained from in silico analysis and design analysis are stored for use in improved analysis of future target sequences, and information about investigators requesting the produced detection assays are stored and used to generate an information template for receiving detection assay data from the user after the assays are used (Section IV). If in silico analysis determines that a SNP was previously characterized, the new request is assessed to see if it provides any additional information (e.g., additional information provided by the new user), and such new information is integrated into the existing records for that SNP in the databases (e.g., association databases, allele frequency databases). In some embodiments, the information about the target sequence and SNP obtained from the in silico, design, and production analysis are integrated with the information template to allow the investigator to access information (e.g., disease associations, allele frequency, etc.) prior to, during, or following use of the detection assay (e.g., information may be linked to a plate viewer function described in Section IV above).

The investigator uses the detection assay on one or more samples, e.g., as described in Section IV, above. Information and data are collected and returned to the systems of the service provider. Information and data obtained by the service provider from use of the detection assay are used for obtaining regulatory approval of clinical products corresponding to successful detection assays and to supplement information databases and improve in silico analysis, assay design, assay production, and future information dissemination to investigators. For example, additional allele frequency information may be obtained from the investigator. This information is used to supplement allele frequency databases. This information may also be used to increase or decrease the number of samples used during production analysis of allele frequency, as certain samples (e.g., samples from particular ethnic groups, disease states, etc.) may be determined to be of limited information content (e.g., redundant) while others represent important, but previously unidentified or unappreciated populations for future analysis of allele frequency testing. Failure data from investigators (e.g., the failure of hybridization probes to hybridize to target sequences in a sample) is used in future in silico and design analysis.

As is clear from the above description, wide-scale use of the systems and methods of the present invention provides solutions to the unmet needs of the fields of bioinformatics and molecular diagnostics and medicine. Each phase of the invention, from target sequence validation and assay design and production to assay use and data collection provides a continuous circle of data generation and improvement. Wide scale use of the systems and methods of the present invention provides for the generation of reliable detection assays for the detection of any target sequence, wherein assays are designed to work for all individuals (e.g., a single assay that works for all individuals or a plurality of assays, each working for a known sub-set of the population). Databases generated using the systems and methods of the present invention provide comprehensive information pertaining to the allele frequency of mutations in one or more populations and the correlations of sequences and gene expression patterns to phenotypes. Thus, in some embodiments, the present invention provides detection assays and corresponding information databases and analysis systems for accurately screening entire populations (e.g., screening all human newborns) for sequences and expression patterns corresponding phenotypes (e.g., disease states, drug responses, etc.). Using the databases of the present invention, a specific sequence, combination of sequences, or expression patterns in an individual may be correlated to proven responses appropriate for the individual (e.g., avoidance of allergens, therapeutic drug treatments, gene therapy, preventive routes or behaviors, etc.).

B. Development of Clinical Detection Assays

As discussed above, of the thousands of markers evaluated using the systems and methods of the present invention, a sub-set of the markers are reliably detected by the detection assays of the present invention. Where a detection assay is shown to reliably detect a marker (e.g., a medically-relevant marker), detections assays for use as analyte-specific reagents or clinical diagnostics are prepared. Analyte-specific reagents and clinical diagnostics are regulated in the United States. Using the systems and methods of the present invention, data generated during the development of the detection assays is used to support regulatory approval of the detection assay for used as analyte-specific reagents and clinical diagnostics. Because the present invention provides easy-to-use, efficient, accurate detection assays (e.g., the INVADER assay) that can be produced for thousands of unique markers at high production capacity and because the present invention provides systems and methods for widespread testing and data collection of thousands of samples with each of the thousands of unique detection assays, sufficient information is gathered to support regulatory approval of numerous clinical products. The present invention provides systems and methods for testing all identified markers, selecting markers that are suitable for clinical use, and collecting data in support of regulatory approval for every clinically relevant marker. The specific regulatory requirements for analyte-specific reagents and in vitro diagnostics are outlined below.

I. Analyte-Specific Reagents

In some embodiments, components of nucleic acid detection assays are sold as analyte specific reagents (ASRs). ASRs are restricted devices under section 520(e) of the Federal Food, Drugs, and Cosmetic Act and 21 CFR 809.30 and are subject to specific restrictions. ASRs may only be sold to “in vitro diagnostic manufacturers”: clinical laboratories regulated under the Clinical Laboratory Improvement Amendments of 1988 (CLIA), as qualified to perform high complexity testing under 42 CFR part 493 or clinical laboratories regulated under VHA Directive 1106 (available from Department of Veterans Affairs, Veterans Health Administration, Washington, D.C. 20420); and organizations that use the reagents to make tests for purposes other than providing diagnostic information to patients and practitioners (e.g., forensic, academic, research, and other nonclinical laboratories). In addition, ASRs must be labeled in accordance with Sec. 809.10(e). Advertising and promotional materials for ASRs must include the identity and purity (including source and method of acquisition) of the analyte specific reagent and the identity of the analyte; the statement for class I exempt ASR's: “Analyte Specific Reagent. Analytical and performance characteristics are not established”; include the statement for class II or III ASR's: “Analyte Specific Reagent. Except as a component of the approved/cleared test (name of approved/cleared test), analytical and performance characteristics are not established”; and must not make any statement regarding analytical or clinical performance.

Any laboratory that develops an in-house test using the ASR is required to inform the ordering person of the test result by appending to the test report the statement: “This test was developed and its performance characteristics determined by (Laboratory Name). It has not been cleared or approved by the U.S. Food and Drug Administration.” This statement would not be applicable or required when test results are generated using the test that was cleared or approved in conjunction with review of the class II or III ASR. Ordering in-house tests that are developed using analyte specific reagents is limited under section 520(e) of the act to physicians and other persons authorized by applicable State law to order such tests.

II. In vitro Diagnostic Detection Assays

In some embodiments, assays for detecting genetic variation are marketed as in vitro diagnostic tests. The marketing of such kits in the United States requires approval by the Food and Drug Administration (FDA). The FDA classifies in vitro diagnostic kits as medical devices. As such, the pre-market applications for most in vitro diagnostics are submitted to the FDA under the 510(k) regulations and are referred to as 510(k) applications. The 510(k) regulations specify categories for which information should be included.

Each person who wants to market Class I, II and some III devices intended for human use in the U.S. must submit a 510(k) to FDA at least 90 days before marketing unless the device is exempt from 510(k) requirements. Classification of devices are determined by finding the regulation number that is the classification regulation for each device. This can be accomplished searching the classification database for a part of the device name, or, if the device panel (medical specialty) to which the device belongs is known, going directly to the listing for that panel and identify the device and the corresponding regulation. Links to both database can be found on the web page of the FDA.

A 510(k) is a premarketing submission made to FDA to demonstrate that the device to be marketed is as safe and effective, that is, substantially equivalent (SE), to a legally marketed device that is not subject to premarket approval (PMA). Applicants must compare their 510(k) device to one or more similar devices currently on the U.S. market and make and support their substantial equivalency claims. A legally marketed device is a device that was legally marketed prior to May 28, 1976 (preamendments device), or a device which has been reclassified from Class III to Class II or I, a device which has been found to be substantially equivalent to such a device through the 510(k) process, or one established through Evaluation of Automatic Class III Definition. The legally marketed device(s) to which equivalence is drawn is known as the “predicate” device(s).

Applicants must submit descriptive data and, when necessary, performance data to establish that their device is SE to a predicate device. The data in a 510(k) is to show comparability, that is, substantial equivalency (SE) of a new device to a predicate device. A claim of substantial equivalence does not mean the new and predicate devices must be identical. Substantial equivalence is established with respect to intended use, design, energy used or delivered, materials, performance, safety, effectiveness, labeling, biocompatibility, standards, and other applicable characteristics.

Once the device is determined to be SE, it can then be marketed in the U.S. If the FDA determines that a device is not SE, the applicant may resubmit another 510(k) with new data, file a reclassification petition, or submit a premarket approval application (PMA). The SE determination is usually made within 90 days and is made based on the information submitted by the applicant.

A 510(k) is required when introducing a device into commercial distribution (marketing) for the first time, when proposing a different intended use for a device which is already in commercial distribution, and when there is a change or modification of a device already marketed that could significantly affect its safety or effectiveness.

Information required in an application under 510(k) includes:

1) The in vitro diagnostic product name, including the trade or proprietary name, the common or usual name, and the classification name of the device.

2) The intended use of the product.

3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified.

4) Proposed labels, labeling and advertisements sufficient to describe the in vitro diagnostic product, its intended use, and directions for use. Where applicable, photographs or engineering drawings should be supplied.

5) A statement indicating that the device is similar to and/or different from other in vitro diagnostic products of comparable type in commercial distribution in the U.S., accompanied by data to support the statement.

6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request.

7) A statement that the submitter believes, to the best of their knowledge, that all data and information submitted in the premarket notification are truthful and accurate and that no material fact has been omitted.

8) Any additional information regarding the in vitro diagnostic product requested that is necessary for the FDA to make a substantial equivalency determination. A request for additional information will advise the 510(k) submitter that there is insufficient information contained in the original 510(k) submission for a substantial equivalent determination to be made. In this situation the 510(k) submitter may: (a) submit the requested data or a new 510(k) containing the requested information, or (b) submit a PMA application in accordance with section 515 of the FD&C Act. If the additional information is not submitted within 30 days following the date of the request, the FDA may consider the 510(k) to be withdrawn.

Factors used by FDA reviewers in determining substantial equivalency include:

1) Does the in vitro diagnostic device have the same intended use as a currently marketed device (sometimes referred to as a “predicate device”), e.g., nucleic acid diagnostic assay?

2) Does the in vitro diagnostic device have the same technological characteristics, e.g., nucleic acid probes?

3) If new technological features are present, e.g., DNA probe, monoclonal antibody, do they raise new questions regarding safety and effectiveness?

Additionally, the following questions will be used by FDA reviewers to assess whether an in vitro diagnostic device that includes technological changes is substantially equivalent to a predicate device.

1) Does the in vitro diagnostic device pose the same type of questions about safety and effectiveness as the predicate device?

2) Are there accepted scientific methods for assessing the impact of technological changes on safety and effectiveness, e.g., accuracy, specificity, sensitivity, precision?

Data generated using the system and methods of the present invention provides sufficient information to obtain approval on the detection assays. Prior to the present invention, only a small number of in vitro diagnostic detection assays have been approved. The present invention provides system and methods for producing approved detection assays for the hundreds of most medically relevant markers. As such, the present invention provides the predicate devices for many markers by which future detection assays will be compared. In some embodiments, the present invention provides methods for obtaining regulatory approval of new detection assays by comparing data obtained with the new detection assay (e.g., data obtained using the systems and methods of the present invention) to a predicate device obtained by using the systems and methods of the present invention.

C. Distribution and Use of Detection Assays

As discussed above, the use of detection assays in the context of research products using the systems and methods of the present invention generates data that finds use in obtaining regulatory approval for clinical products and in the generation of databases. In some embodiments of the present invention, a party with interest in selling clinical products or information stored in databases provides (e.g., using any delivery systems) detection assays to researchers in order to collect data. In some embodiments, the party provides detection assays to researchers at a reduced cost, at a subsidized cost, or at no cost. In yet other embodiments, the party pays a researcher to use the test in order to gain access to data obtained from the test. Using the systems and methods of the present invention, the party can compensate for any lost profits or revenues by obtaining and selling clinical products, which are typically high revenue, high margin products.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method of validating a detection assay, comprising:
a) distributing one or more detection panels to a plurality of users, wherein said detection panels comprise a plurality of candidate detection assays configured for target detection;
b) collecting test result data from at least a portion of said plurality of users, wherein said test result data is generated with said detection panels; and
c) processing at least a portion of said test result data such that at least one valid detection assay is identified from said plurality of candidate detection assays

2. The method of claim 1, further comprising step d) marketing said valid detection assay as an Analyte-Specific Reagent or an In-Vitro Diagnostic.

3. The method of claim 1, wherein said plurality of detection assays comprise two or more unique detection assays.

4. The method of claim 1, further comprising a distribution system, wherein said distributing is accomplished with said distribution system.

5. The method of claim 1, wherein said distributing one or more detection panels to said plurality of users is at a reduced cost.

6. The method of claim 1, wherein said distributing one or more detection panels to said plurality of users is at a subsidized cost.

7. The method of claim 1, wherein said distributing one or more detection panels to said plurality of users is at no cost.

8. The method of claim 1, wherein prior to step a), the method further comprises the step of employing one or more of said plurality of candidate detection assays to discover at least one single nucleotide polymorphism.

9. The method of claim 8, wherein said plurality of detection assays comprise INVADER assays.

10. The method of claim 1, wherein prior to step a), the method further comprises the step of utilizing one or more of said plurality of candidate detection assays to associate a single nucleotide polymorphism with a medical condition.

11. The method of claim 10, wherein said plurality of detection assays comprise INVADER assay components.

12. The method of claim 1, wherein prior to step a), the method further comprises the step of utilizing one or more of said plurality of candidate detection assays, and bioinformatic analysis, to associate a single nucleotide polymorphism with a medical condition.

13. The method of claim 1, wherein said plurality of detection assays comprise INVADER assay components.

14. The method of claim 13, wherein said INVADER assay components comprise an INVADER oligonucleotide, a probe, and a control target sequence.

15. The method of claim 1, wherein said plurality of detection assays comprise TAQMAN assay components.

16. The method of claim 15, wherein said TAQMAN assay components comprise a probe.

17. The method of claim 1, wherein said one or more detection panels are configured for detecting a marker associated with a disease category.

18. The method of claim 17, wherein said disease category is selected from cardiovascular disease, cancer, autoimmune disease, metabolic disorders, neurological disease, musculoskeletal disorders, and endocrine related diseases.

19. The method of claim 1, wherein said plurality of users comprises researchers.

20. The method of claim 1, wherein said plurality of users comprises 10 individual users.

21. The method of claim 1, wherein said plurality of users comprises at least 200 individual users.

22. The method of claim 1, wherein said plurality of users comprises at least 500 individual users.

23. The method of claim 1, wherein said plurality of users comprises at least 1000 individual users.

24. The method of claim 1, wherein said plurality of users comprises at least 10,000 individual users.

25. The method of claim 1, wherein said plurality of detection assays comprises at least 10 unique detection assays.

26. The method of claim 1, wherein said plurality of detection as says comprises at least 1000 unique detection assays.

27. The method of claim 1, wherein said plurality of detection assays comprises at least 10,000 unique detection assays.

28. The method of claim 1, wherein said plurality of detection assays comprises at least 50,000 unique detection assays.

29. The method of claim 1, further comprising a step, after said processing step, of selling said at least one valid detection assay as an Analyte Specific Reagent (ASR).

30. The method of claim 1, further comprising a step, after said processing step, of selling said at least one valid detection assay as an Analyte Specific Reagent (ASR) to an In-Vitro Diagnostic Manufactuer or to a non-clinical laboratory.

31. The method of claim 1, further comprising a step, after said processing step, of selling said at least one valid detection assay as an In-Vitro Diagnostic.

32. The method of claim 1, wherein said test result data comprises raw assay data.

33. The method of claim 1, wherein said test result data comprises analyzed assay data.

34. The method of claim 1, wherein said test result data comprises data resulting from testing of 110,000 separate samples.

35. The method of claim 1, wherein said collecting comprises receiving said test result data from at least a portion of said plurality of users over a communications network.

36. The method of claim 35, wherein said collecting further comprises storing said test result data in a database.

37. The method of claim 36, wherein said database is part of a computer system of a service provider.

38. The method of claim 1, wherein said collecting comprises receiving said test result data over the Internet.

39. The method of claim 1, wherein said collecting comprises retrieving said test result data from a user's computer system over a communication network.

40. The method of claim 39, wherein said user's computer system comprises a software application configured to receive said test result data.

41. The method of claim 40, wherein said software application is further configured to transmit said test result data automatically via a communications network.

42. The method of claim 1, wherein said processing comprises categorizing said test result data.

43. The method of claim 1, where said processing comprises computer aided data analysis of said test result data.

44. The method of claim 1, wherein said processing comprises mathematical manipulation of said test result data.

45. The method of claim 1, wherein said processing comprises comparing said test result data to a substantially equivalent predicate assay.

46. The method of claim 1, wherein said processing comprises mathematical manipulation of said test result data, and comparing said test result data to a substantially equivalent predicate assay.

47. The method of claim 1, wherein at least one valid detection assay is identified as a result of being substantially equivalent to a predicate assay.

48. The method of claim 1, wherein said processing at least a portion of said test result data generates assay validation information.

49. The method of claim 48, further comprising step e) submitting said assay validation information to a government body charged with approving products for clinical use.

50. The method of claim 49, wherein said government body is the Food and Drug Administration.

51. The method of claim 49, wherein said assay validation information is part of a 510(k) application that is submitted to the Food and Drug Administration.

52. The method of claim 50, further comprising a step of receiving approval from said Food and Drug Administration to market said at least one valid detection assay as an FDA approved In-Vitro diagnostic assay.

53. The method of claim 52, wherein said FDA approved In-Vitro diagnostic assay is a predicate for determining substantially equivalency for other In-Vitro diagnostic assays.

54. The method of claim 1, wherein said target comprises a DNA molecule

55. The method of claim 1, wherein said target comprises a RNA molecule.

56. A method of developing an in-vitro diagnostic DNA or RNA analysis product comprising, running an assay through a product development funnel, in which said assay that enters said product development funnel is substantially similar to said in-vitro diagnostic DNA or RNA analysis product.

57. The method of claim 57 in which said assay is an assay to detect a single nucleotide polymorphism.

58. The method of claim 58 in which said product development funnel optionally comprises a discovery portion.

59. The method of claim 59 in which said product development funnel optionally comprises a medically associated portion.

60. The method of claim 60 in which said product development funnel optionally comprises an analyte-specific reagent portion.

61. The method of claim 61 in which said product development funnel optionally comprises an in-vitro diagnostic portion.

62. The method of claim 59 in which said assay comprises a chromosome specific assay.

63. The method of claim 59 further comprising using a panel and in which said panel comprises said assay.

64. The method of claim 64 in which said panel comprises a whole genome panel

65. The method of claim 60 in which said medically associated portion comprises a panel organized by disease.

66. The method of claim 66 in which said panel organized by disease is selected from the group consisting of a cardiovascular disease panel, an oncology panel, an immunology panel, a metabolic disorders panel, a neurological disorders panel, a musculoskeletal disorders panel, an endocrinology panel, and a genetic disease panel.

67. The method of claim 60 further comprising using a panel, and in which said panel is a panel for a multiplicity of disease states.

68. The method of claim 60 further comprising using a panel, and in which said panel comprises a drug metabolizing enzyme panel.

69. A method of increasing revenue and/or a profit margin from the development of an in-vitro diagnostic DNA or RNA analysis product comprising channeling an assay through a product development funnel, in which said assay is substantially similar to said in-vitro diagnostic DNA or RNA analysis product.

70. The method of claim 70 in which said in-vitro DNA or RNA analysis product comprises an FDA approved product.

71. The method of claim 70 in which said product development funnel has an ingress and an egress, and in which said assay is one of at least several thousand assays which enter said ingress.

72. The method of claim 72 in which said assay is one of about several hundred assays which exit said egress as said in-vitro diagnostic DNA or RNA analysis product.
